(19) United States
(12) Patent Application Publication
(10) Pub. No.: US 2020/0385777 A1
(43) Pub. Date: Dec. 10, 2020

(54) METHOD FOR DETERMINING DYSBIOSIS IN THE INTESTINAL MICROBIOME

(71) Applicant: EVOLVE BIOSYSTEMS, INC., Davis, CA (US)

(72) Inventors: David KYLE, Davis, CA (US); Steven FRESE, Oakland, CA (US); Samara FREEMAN-SHARKEY, Davis, CA (US); Bethany HENRICK, Davis, CA (US)

(21) Appl. No.: 16/959,595

(22) PCT Filed: Jan. 3, 2019
(86) PCT No.: PCT/US2019/012229

§ 371 Date: Jul. 1, 2020

Related U.S. Application Data
(60) Provisional application No. 62/613,405, filed on Jan. 3, 2018.

(51) Int. Cl. C12Q 1/04; G01N 21/79; G01N 21/80; G01N 33/68

(52) U.S. Cl. CPC C12Q 1/04; G01N 33/6863; G01N 21/80; G01N 21/79

(57) ABSTRACT

The inventions described herein relate generally to the methods for monitoring the health of the mammalian gut by checking for whether dysbiotic parameters exceed a threshold level or not. In particular, this invention is directed to the use of parameters which correlate with the level of bifidobacteria, especially Bifidobacterium longum subsp. infantis in the mammalian colon.

### FIELD OF INVENTION

The inventions described herein relate generally to the methods for monitoring the health of the mammalian gut by checking for whether certain parameters exceed a dysbiotic threshold level or not. In particular, this invention is directed to the use of parameters which correlate with the total level of bifidobacteria, and/or the status of specific species such as Bifidobacterium longum subsp. infant's, in the mammalian colon.

### BACKGROUND

The intestinal microbiome is the community of microorganisms that live within an animal's gastrointestinal tract, in mammals the vast majority are found in the large intestine or colon. In a healthy human, most dietary carbohydrates that are consumed are absorbed by the body before they reach the colon. Many foods, however, contain indigestible carbohydrates (i.e., dietary fiber) that remain intact and are not absorbed during transit through the gut to the colon. The non-infant or adultcolonic microbiome is rich in bacterial species that may be able to fully or partially consume these fibers and utilize the constituent sugars for energy and metabolism creating different metabolites for potential nutritive use in the mammal. The adult mammalian microbiome is complex and contains a diverse community of species of bacteria. Conventional teaching with regards to the non-infant mammalian microbiome is that complexity provides stability, and maintaining a diversity of microorganisms in the microbiome while consuming a complex diet is thought to be the key to promoting gut health. Lozupone, Nature, Vol. 489, pp. 220-230 (2012). Methods for measuring dietary fiber in various foods are well known to one of ordinary skill in the art.

The nursing human infant's intestinal microbiome is quite different from an weaned infant, toddler, child or adult (non-infant) microbiome in that the adult gut microbiome generally contains a large diversity of organisms, each present at a low percentage of the total microbial population. By comparison, a healthy infant gut is far less diverse with a single species dominating the microbiome. Further, infant nutrition is typically limited to a single nutrient source, mother's milk, and dietary fiber in an infant's colon is likewise limited. Mammalian milk contains a significant quantity of mammalian milk oligosaccharides (MMO) as dietary fiber. For example, in human milk, the dietary fiber is about 15% of total dry mass, or about 15% of the total caloric content. These oligosaccharides comprise sugar residues in a form that is not usable directly as an energy source for the baby or an adult, or for most of the microorganisms in the gut of that baby or adult. In healthy infants, all dietary fiber may be consumed by a single bacterial species [Locascio, 2010 Appl Environ Microbiol. 2010 November; 76(22):7373-81]. Consequently, the infant microbiome is typically quite simple. The healthy nursing infant's microbiome can be made up almost exclusively of a single species that may represent at least 60-80% of the total number of species that make up the infant gut microbiome. When this species is B. infantis and the infant is a human infant, this dominant colonization unexpectedly gives rise to a very stable gut ecology [Frese, 2017 mSphere 2:e00501-17. https://doi.org/10.1128/mSphere 0.00501-17]. Microbiome stability is a desirable characteristic in the first few months of life where many developmental changes are rapidly taking place as the infant develops prior to weaning.

The complexity of the adult microbiome begins to develop after the cessation of human milk consumption as a sole source of nutrition. The transition from the simple, non-diverse microbiome of the nursing infant to a complex, diverse adult-like microbiome (i.e., weaning) correlates with the transition from a single nutrient source of a rather complex fiber (e.g., maternal milk oligosaccharides) to more complex nutrient sources that have many different types of dietary fiber.

### SUMMARY OF INVENTION

Creating a healthy microbiome in a mammal is necessary for the proper health of the mammal and to avoid dysbiosis. While it is difficult to understand the exact makeup of the microbiome at any given time in a mammal, the inventors have found observable signals of dysbiosis or health of the infant microbiome in the stool composition, biochemistry, pH and other stool biomarkers. The presence of certain amounts of organic acids and short-chain fatty acids (SCFA) in the stool of a mammal and more specifically lactate and acetate, can be a signal of a healthy microbiome or their lack results in a dysbiosis that needs to be corrected. The inventors have discovered that the increase of certain microbes under a controlled diet of mammalian milk oligosaccharides will result primarily in the increase of lactate and acetate; furthermore these certain microbes can account for the majority of the observed increase in organic acid and SCFA in the colon and decrease in pH. The parameters for this invention can be used to provide a readout on the status of the intestinal microbiome using a threshold level below or above which one can infer that the intestinal microbiome is healthy or dysbiotic.

This invention provides a method of monitoring the status of a mammal's gut microbiome as it relates to dysbiosis and provide a readout useful in assessing overall health as it relates to digestive discomfort including diarrhea, colic, fussiness, excessive crying, risk of acute infections, (e.g. risk of infection from potential pathogens, increased presence of antibiotic resistant genes, risk of antibiotic resistant infections) and/or inappropriate immune development or chronic inflammation states that may increase risk of future disease (e.g. atopy, obesity, allergy, necrotizing enterocolitis), by obtaining a fecal sample from the mammal; determining the level of at least one dysbiotic parameter in the fecal sample; and determining whether the level the dysbiotic parameter(s) exceeds a threshold, where exceeding said threshold provides a signal reflective of dysbiosis in the mammal. Indicators suitable for this invention include titratable acidity or total acidity, relative amount of low molecular weight organic acids including short-chain fatty acids (SCFA), in particular lactic acid and acetic acid, SCFA content, pH, amount of total bifidobacteria, amount of B. infantis, amount of pathogenic bacteria, amount of lipopolysaccharide (LPS), amount of antibiotic resistance genes, amount of human milk oligosaccharides (HMO), or other mammalian milk oligosaccharides (MMO), amount of inflammatory markers. inflammatory markers may include cytokines, expression of receptors in immune mediated pathways, polymorphonuclear cell infiltration, production of protein biomarkers such as calprotectin, and/or production of innate immune factors consistent with inflammation, such as but not limited to Soluble Toll like receptor 2 (sTLR2), soluble CD83 (SCD83 or, soluble CD14 (SCD14).

Threshold levels of the dysbiotic parameter may be (a) lactate:acetate ratio of less than 0.55 in the feces by mole; (b) cytokines (e.g., IL1 beta, Il-2, IL-5, IL-6, IL-8 and IL-10, IL-22, INF-gamma and/or TNF-alpha), innate immune factors (e.g., soluble (s) Cluster of Differentiation (CD)14 and sCD83), soluble Toll-like Receptors (sTLR2, sTLR4), calprotectin, and/or C-reactive protein (CRP) at least 2× the level found in the feces of infants having greater than 10⁸ CFU B. infantis/g feces; (c) LPS at least 2× the level found in the feces of infants having greater than 10⁸ CFU Bifidobacterium/g feces; (d) pathogenic bacteria levels at least 4× higher in the feces, compared to infants having greater than 10⁸ CFU Bifidobacterium/g feces; (e) antibiotic resistance gene load (e.g., number of antibiotic resistance genes (ARGs), ARG expression level, ARG diversity) at least 3× higher in the feces, compared to infants having greater than 10⁸ CFU B. infantis/g feces; (f) organic acid content (e.g., lactate and acetate) at least a decrease of 10 μmol/g feces, preferably 20 μmol/g feces, compared to infants having greater than 10⁸ CFU Bifidobacterium/g feces and/or a threshold of at least 30 μmol/g feces; (g) bifidobacteria levels of less than 10⁸ CFU/g, preferably less than 10⁷, more preferably less than 10⁶ in the feces; (h) B. infantis levels of less than 10⁸ CFU/g, preferably less than 10⁷, more preferably less than 10⁶ in the feces; (i) increased HMO levels present in the feces of at least an order of magnitude, compared to infants having greater than 10⁸ CFU B. infantis/g feces, and/or a threshold of greater than 10 mg/g of feces; (j) pH equal to or greater than 5.85; and/or (k) a Jaccard stability index (JSI) lower than 0.5. (k) one or more of the following cytokines (pg/gram feces) have a threshold that is cytokine specific: IL-8 is greater than or equal to than 114; TNF-alpha greater than 6, INF-gamma greater than 51; IL-1beta is greater than 43; IL-22 is greater than 3; IL-2 is greater than 4; IL-5 is greater than 3; IL-6 is greater than 1; and IL-10 is greater than 1. Pathogenic bacteria determined according to this invention may be identified at the family, genus or species level and can include members of the Enterobacteriaceae family (e.g., Salmonella, E. coli, Klebsiella, Cronobacter), members of the family Clostridiaceae/class Clostridia (e.g., Clostridium difficile), or Bacteroidaceae family/Bacteroides genus. or combinations thereof. At least one of certain species of pathogenic bacteria may be monitored including but not limited to Klebsiella pneumonia, Enterobacter cloacae, Staphylococcus aureus, Staphylococcus epidermidis and Clostridium perfringens. SCFA measured according to this invention may include one or more of formic, acetic, propionic, and butyric acids and salts thereof, and lactic acid or salts thereof. oIn some embodiments, one or more cytokines may be considered when determining dysbiosis. In one embodiment the level above the threshold is considered specifically for IL-8, 11-10 and TNF-alpha; in other embodiments, IL-1B. INFgamma and TNF-alpha are considered together to determine presence or absence of dysbiosis. In yet other embodiments, the threshold for a particular cytokine or group of cytokines is determined based on the age of the infant (eg. the threshold of a particular cytokine at day 40 of life may be different from the threshold at 60 days and require a different action). In some embodiments the threshold is age adjusted to determine dysbiois. In further embodiments, the threshold for insufficient Bifidobacterium is determined by inflammatory markers above their respective thresholds. In some embodiments less than 2%, less than 30% or less than 40% may indicate dysbiosis.

Mammals whose health is monitored according to this invention may include human or non-human mammals, where the non-human mammal may be a buffalo, camel, cat, cow, dog, goat, guinea pig, hamster, horse, pig, rabbit, sheep, monkey, mouse, or rat, and the non-human mammal may be a mammal grown for human consumption, or a companion or performance animal. The mammal may be a human infant, either a pre-term infant or a term infant, particularly an infant born by C-section.

In particular embodiments, this invention provides a method of determining the level of Bifidobacterium in a mammal by measuring titratable acidity in a fecal sample, the method comprising the steps of: (a) mixing a predetermined amount of a mammalian fecal sample with a fixed amount of NaOH at a ratio of 10 μmol/g fecal sample, (b) adding an ethanol solution containing 1% phenolphthalein to provide phenolphthalein indicator in the mixture, and (c) monitoring the color of the resultant mixture, where mixtures that stay fuchsia or pink may be recognized to come from mammals having low bifidobacteria in their colon, and mixtures that change their color away from fuchsia/pink towards yellow/peach may be recognized as having come from mammals having high bifidobacteria levels in their colon. In preferred embodiments, the fecal sample is from a human infant. This embodiment is useful for monitoring the intestinal condition of a human infant for the prevention or treatment of dysbiosis.

Methods of this invention can be used to establish a baseline intestinal state for a newborn mammal, including, but not limited to a human infant, a foal, or a pig by using one or more dysbiotic signals as a single point in time or in monitoring over time. It can also be used to monitor the status of any intervention related to providing prebiotic, probiotics, or probiotic plus prebiotic combinations to a mammal to establish the effectiveness of said intervention on improving the status of one or more dysbiotic signals. It can also be used to inform a course of treatment for a mammal. It can be used to specifically monitor total Bifidobacterium and/or B. infantis levels or colonization of the mammalian colon. In some embodiments, the method is a point of care test, a near point of care test, and/or a lab test.

### BRIEF DESCRIPTION OF THE FIGURES

FIG. 1. Amount (CFU/g) of B. longum subsp. infantis (B. infantis) in fecal samples as measured by qPCR during the intervention period and a follow-up period in both vaginally- and C-section-delivered human infants. The black line and dots represent all infants who were supplemented with B. infantis for 21 days starting at 7 days of life. All infants receiving the standard of care (no probiotic) are depicted with the grey line and dots. The band around each line represents a 95% confidence interval around the line. The end of supplementation occurred at day 28 and samples were collected until day 60 of life.

FIG. 2A. Abundances of different genera of intestinal bacteria in an untreated C-section baby over the study period (Day 6 to 60 of life).

FIG. 2B. Abundance of different genera of intestinal bacteria in a C-section baby treated from Day 7 to 28 with B. longum subsp. infantis.

FIG. 3. Predictive antibiotic (AB) resistance gene load in fecal samples taken from unsupplemented (white bars) or supplemented (black bars) infants.

FIG. 4. Mean concentration of fecal HMO (+/−SD, mg/g) in infant stools collected at baseline (Day 6; pre-supplementation) and at the end of supplementation (Day29; post-supplementation). Dark grey bars represent the B. infantis supplemented group.

FIG. 5. Box plot of endotoxin levels (Log EU/ml) in fecal samples from unsupplemented infants devoid of all bifidobacteria (Bifidobacterium-naïve) vs. fecal samples from infants supplemented with B. infantis and replete with bifidobacteria (High Bifidobacteria).

FIG. 6. Hierarchical clustering based on strain-level analysis of Bifidobacterium longum subspecies. Gene family profiles of a subgroup of reference genomes were selected from a global (n=38) strain analysis. Each column represents presence or absence of genes in a sample or a reference genome in respect to the total pangenome. All EVC001 samples clustered together with B. longum ssp. infantis ATCC 15697 (B. infantis) showing identical profiles, while control samples clustered separately with different B. longum subspecies (e.g., B. suis, B. longum DJ01A, B. longum NCC2705). Functional analysis of gene families confirmed that EVC001 samples were dominated by B. infantis due to the presence of unique key genetic clusters (e.g., HMO-cluster 1), while missing genes known to be present only in B. longum ssp. longum (e.g., araD; araA), which were only present in the control community. P-values bar for every gene was computed via Fisher's exact test.

FIG. 7. Relative abundance of total resistome profile in each metagenomics sample. A) Relative abundance of ARGs compared to overall metagenome for every sample. Every dot represents a sample resistome (control=31; EVC001=29). Box plots on the right denote the interquartile range (IQR), with horizontal lines representing the 25th percentile, median, and 75th percentiles. The whiskers represent the lowest and highest values within 1.5 times the IQR from the first and third quartiles, respectively. The asterisks on the top indicate significant P28 values (Mann-Whitney test). B) Relative abundance of bacterial genera in the overall metagenome assigned to antibiotic resistance genes. Shade of colors represents genera belonging to the same bacterial class. The asterisks on the top indicate significant P-values (Kruskal-Wallis test).

FIG. 8 Comparison of the most significant antibiotic resistance gene types. A) Relative abundance of the top (n=38) most significant antibiotic resistance genes (ARGs) identified among EVC001-supplemented infants and controls. Percentages are relative to overall metagenomic content. These ARGs are known to confer resistance to different drug classes including beta-lactams, fluoroquinolones, and macrolides. ARGs are grouped by color according to drug class (legend). B Heatmap showing hierarchical cluster analysis of total identified ARGs (n=652) among samples. Two main clusters were produced, the right panel (whiter), characterized by a lower-ARG carriage and the left panel (red) by a higher carriage. The majority of EVC001-supplemented samples, clustered within the lower panel, with few controls, which had in common natural delivery mode and a lower level of Enterobacteriaceae family. Higher levels of Bifidobacteria (e.g. B. infantis) were associated with a lower abundance of ARGs, whereas higher levels of gram negative bacteria (e.g. Escherichia) were related with an increased abundance of ARGs. Genes clustered based on similar biological mechanisms implicated in drug resistance (see Results). P-values on the bar were computed using Kruskal-Wallis test normalized with Bonferroni correction. On the right of the heatmap, the respective P-values are color-coded by significance for any of the ARGs identified. The top of the heatmap shows hierarchical separation of EVC001 vs Control samples based on overall resistome profile. Finally, all the individual families relative abundance is shown on the bottom of the heatmap.

FIG. 9. Quantification of Enterobacteriaceae family by group specific qPCR. The data are represented as the mean Log 10 CFU per gram of stool sample+/−SEM (***P<0.0001, Mann-Whitney Test).

FIG. 10. Diversity analyses of infant resistomes according to probiotic supplementation with EVC001. A) Rarefaction curves showing number of unique antibiotic resistance genes (ARGs) identified in relation to the increasing number of sequences. Both EVC001 and the control group presented similar curve trends, suggesting that sequencing depth is not associated with the diversity of antibiotic resistance. The EVC001 group reported less than half unique ARGs compared to the control samples. P-value was computed with a nonparametric two-sample t-test using Monte Carlo permutations (n=999). B) Global resistome profiles computed via principal coordinate analysis (PCoA) based on Bray-Curtis dissimilarity matrix. EVC001 samples clustered closely, indicating a much more similar resistome profiles compared to the controls, which had a more disperse distribution. The effect of colonization by B. infantis EVC001 itself accounted for 31% of the total explained variation (adonis). P-value was computed using F-tests based on sequential sums of squares from permutations of the raw data.

FIG. 11. Correlation between relative abundance of bacterial families and fecal pH. Bacterial families identified via 16S rRNA marker gene sequencing significantly correlated with fecal pH. Lower pH was strongly and uniquely correlated with greater Bifidobacteriaceae abundance (r=−0.4; p<0.001**; Spearman). Higher pH was significantly correlated with the Clostridiaceae, Enterobacteriaceae, Peptostreptococcaceae and Veillonellaceae families. P-values are represented by asterisks (*, P<0.05; **, P<0.01; ***, P<0.001; ****, P<0.0001)

FIG. 12. Fecal samples from healthy, breastfed infants were evaluated for relative abundance of Bifidobacterium using qPCR. Data indicated a bimodal distribution in which fecal samples either had high or low Bifidobacterium abundance.

FIG. 13. (A) Mean fecal pH (±SD) at day 21 from infants with no Bifidobacterium, Bifidobacterium species except infantis, or B. infantis. (B) Mean organic acids (acetate and lactate) in fecal samples at day 21 postpartum with no Bifidobacterium, Bifidobacterium species except infantis, or B. infantis alone. P-values are represented by asterisks (*, P<0.05; **, P<0.01; ***, P<0.001; ****, P<0.0001)

FIG. 14. Fecal Bifidobacterium counts (log₁₀ cells per gram feces) correlate with pH.

FIG. 15. Temporal changes in 3 key cytokines expressed in pg/gram of feces. Left bars represent unsupplemented infants; right bars represent EVC001 fed infants. (A) Measurement of TNFalpha at Day 6, 40, and 60; (B) Measurement of IL-8 at Day 6, 40, and 60; and (C) Measurement of IL-10 at Day 6, 40, and 60.

FIG. 16. Determination of fecal Calprotectin levels in stool samples taken at Day 40. (A) difference in fecal calprotectin in samples with less than 2% Bifidobacterium (B) fecal caprotectin levels vs relative abundance of Bifidobacteriaceae; (C) Bifidobacterium dysbiosis as a marker for atopy risk.

### DETAILED DESCRIPTION OF THE INVENTION

This invention is directed to methods of monitoring dysbiosis or microbiome function, particularly by determining whether one or more parameters measured in mammalian feces exceed a threshold level, where the parameter is correlated with the level of bifidobacteria colonizing the colon of the mammal.

Definition of Dysbiosis

Generally, the phrase “dysbiosis” describes a non-ideal state of the microbiome inside the body, typified as an insufficient level of keystone bacteria (e.g., bifidobacteria, such as B. longum subsp. infantis) or an overabundance of harmful bacteria in the gut. Dysbiosis can be further defined as inappropriate diversity or distribution of species abundance for the age of the human or animal. Dysbiosis may also refer to the abundance of specific gene functions, such as, but not limited to abundance of antibiotic resistance genes in the microbiome. Dysbiosis, in a human infant is defined herein as a microbiome that comprises total Bifidobacterium and more specifically B. longum subsp. infantis below the level of 10⁸ CFU/g fecal material during the first 6-12 months of life, likely below the level of detectable amount (i.e., ≤10⁶ CFU/g fecal material).

Conversely, the phrase “healthy”, “non-dysbiotic is taken to mean a microbiome that has sufficient levels of keystone bacteria, likely above the level of 10⁸ CFU/g fecal material, and a lower level of pathogenic bacteria, likely below the level of detectable amount (i.e., 10⁶ CFU/g fecal material).

Definition of Mammalian Milk Oligosaccharide

The term “mammalian milk oligosaccharide” or MMO, as used herein, refers to those indigestible glycans found in mammalian milk, sometimes referred to as “dietary fiber”, or the carbohydrate polymers that are not hydrolyzed by the endogenous mammalian enzymes in the digestive tract (e.g., the small intestine) of the mammal. Mammalian milks contain a significant quantity (i.e. g/L) of MMO that are not usable directly as an energy source for the milk-fed mammal but may be usable by many of the microorganisms in the gut of that mammal. The oligosaccharides (3 sugar units or longer, e.g., 3-20 sugar residues) that make up the MMOs, can be found free or they may be conjugated to proteins or lipids. Oligosaccharides having the chemical structure of the indigestible oligosaccharides found in any mammalian milk are called “MMO” or “mammalian milk oligosaccharides” herein, whether or not they are actually sourced from mammalian milk. MMO includes human milk oligosaccharides.

Particular oligosaccharides that may be found in MMO include, but are not limited to fucosyllactose, lacto-N-fucopentose, lactodifucotetrose, sialyllactose, disialyllactone-N-tetrose, 2′-fucosyllactose, 3′-sialyllactoseamin, 3′-fucosyllactose, 3′-sialyl-3-fucosyllactose, 3′-sialyllactose, 6′-sialyllactosamine, 6′-sialyllactose, difucosyllactose, lacto-N-fucosylpentose I, lacto-N-fucosylpentose II, lacto-N-fucosylpentose III, lacto-N-fucosylpentose V, sialyllacto-N-tetraose, or derivatives thereof. See, e.g., U.S. Pat. Nos. 8,197,872, 8,425,930, and 9,200,091, the disclosures of which are incorporated herein by reference in their entirety. The major human milk oligosaccharides (“HMO”), include lacto-N-tetraose (LNT), lacto-N-neotetraose (LNnT) and lacto-N-hexaose, which are neutral HMOs, in addition to fucosylated oligosaccharides such as 2-fucosyllactose (2FL), 3-fucosyllactose (3FL), and lacto-N-fucopentaoses I, II and III. Acidic HMOs include sialyllacto-N-tetraose, 3′ and 6′ sialyllactose (6SL). HMO are particularly highly enriched in fucosylated oligosaccharides (Mills et al., U.S. Pat. No. 8,197,872). These oligasaccharides may be consumed or metabolized by the bacteria in the microbiome of a heathy infant, or they may pass through the colon and into the feces of a dysbiotic infant.

Microbes of the Healthy Newborn Microbiome

Certain microorganisms, such as Bifidobacterium longum subsp. infantis (B. infantis), have the unique capability to consume specific MMO, such as those found in human (HMO) or bovine (BMO) milk (see, e.g., U.S. Pat. No. 8,198,872 and U.S. patent application Ser. No. 13/809,556, the disclosures of which are incorporated herein by reference in their entirety). When B. infantis comes in contact with certain MMO, a number of genes are specifically induced which are responsible for the uptake and internal deconstruction of those MMO, and the individual sugar components are then catabolized to provide energy for the growth and reproduction of that microorganism (Sela et al., 2008). This form of carbon source utilization is remarkably different from most of the other colonic bacteria, which produce and excrete extracellular glycolytic enzymes that deconstruct the fiber to monomeric sugars extracellularly, and only monomers are imported via hexose and pentose transporters for catabolism and energy production.

Total Bifidobacterium, B. longum or more specifically B. longum subsp. infantis, can be monitored to assess the state of dysbiosis or the lack of dysbiosis (healthy state). The beneficial bacteria monitored can be a single bacterial species of Bifidobacterium such as B. adolescentis, B. animalis (e.g., B. animalis subsp. animalis or B. animalis subsp. lactis), B. bifidum, B. breve, B. catenulatum, B. longum (e.g., B. longum subsp. infantis or B. longum subsp. longum), B. pseudocatanulatum, B. pseudolongum, single bacterial species of Lactobacillus, such as L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius, L. paracasei, L. kisonensis., L. paralimentarius, L. perolens, L. apis, L. ghanensis, L. dextrinicus, L. shenzenensis, L. harbinensis, or single bacterial species of Pediococcus, such as P. parvulus, P. lolii, P. acidilactici, P. argentinicus, P. claussenii, P. pentosaceus, or P. stilesii, or it can include and combination of two or more of the species listed here simultaneously or in parallel.

Dysbiotic Microbiome

Dysbiosis in infants is driven by either the absence of MMO, the absence of B. infantis, or the incomplete or inappropriate breakdown of MMO. If the appropriate gut bacteria are not present (e.g., a consequence of the extensive use of antibiotics or cesarean section births), or the appropriate MMO are not present (e.g., in the case of using artificial feeds for newborns, such as infant formula or milk replacers), any free sugar monomers cleaved from the dietary fiber by extra cellular enzymes can be utilized by less desirable microbes, which may give rise to blooms of pathogenic bacteria and symptoms such as diarrhea resulting therefrom. Additionally, the infant mammal may have an increased likelihood of becoming dysbiotic based on the circumstances in the environment surrounding the mammal (e.g., an outbreak of disease in the surroundings of the mammal, antibiotic administration, formula feeding, cesarean birth, etc.).

Dysbiosis in a mammal, especially an infant mammal, can be observed by the physical symptoms of the mammal (e.g., diarrhea, digestive discomfort, colic, inflammation, etc.), and/or by observation of the presence of intact MMO, an abundance of extracellular free sugar monomers in the feces of the mammal, an absence or reduction in specific bifidobacteria populations, and/or the overall reduction in measured organic acids; more specifically, acetate and lactate. Dysbiosis in an infant mammal can further be revealed by a low level of SCFA in the feces of said mammal.

For an infant human, an insufficient level of keystone bacteria (e.g., bifidobacteria, such as B. longum subsp. infantis) may be at a level below which colonization of the bifidobacteria in the gut will not be significant (for example, around 10⁶ CFU/g stool or less). Conversely, certain genus and species of harmful or less desirable bacteria can be monitored. For non-human mammals, dysbiosis can be defined as the presence of members of the Enterobacteriaceae family at greater than 10⁶, or 10⁷, or 10⁸ CFU/g feces from the subject mammal. Additionally, a dysbiotic mammal (e.g., a dysbiotic infant) can be defined herein as a mammal having a fecal pH of 5.85 or higher, a watery stool, Clostridium difficile levels of greater than 10⁶ CFU/g feces, greater than 10⁷ CFU/g feces, or greater than 10⁸ CFU/g feces, Enterobacteriaceae at levels of greater than 10⁶, greater than 10⁷, or greater than 10⁸ CFU/g feces, and/or a stool pH of 5.5 or above, 6.0 or above, or 6.5 or above. For example, a dysbiotic human infant can be a human infant having a watery stool, Clostridium difficile levels of greater than 10⁶ CFU/g feces, greater than 10⁷ CFU/g feces, or greater than 10⁸ CFU/g feces, Enterobacteriaceae at levels of greater than greater than 10⁶, greater than 10⁷, or greater than 10⁸ CFU/g feces, a stool pH of above 5.5, above 5.85 or above, 6.0 or above, or 6.5 or above, lactate:acetate ratios of less than 0.55, and/or organic acid content less than 35 μmol, less than 30 μmol, less than 25 μmol organic acid/g feces, or a reduction in organic acid of at least 10 μmol/g, or at least 20 μmol/g.

The inventors have discovered that the dysbiotic state in an infant can be altered by providing a probiotic and a prebiotic, especially isolated, purified, and activated B. infantis (that specifically consume human milk oligosaccharides) along with human milk oligosaccharides. The increase in total Bifidobacterium resulted in higher levels of SCFA, and in particular increased production of acetic and lactic acids in the feces of that infant mammal, as well as a decrease in fecal pH. The inventors further found that this treatment also significantly lowered the levels of proinflammatory biomarkers as well as pathogenic bacteria and lipopolysaccharide (LPS). Similar observations found in humans, horses, and pigs indicate that this may be a common element among many species that provide milk as the sole source of nutrition for their infant during the first stages of life (i.e., all mammals). These observations are the basis for developing thresholds for distinguishing a dysbiotic state from a healthy state.

Each of the observations identified parameters which were correlated with the state of the microbiome with respect to dysbiosis. Particular parameters were found to exhibit bimodal distribution corresponding to (a) healthy infants colonized with high levels of total Bifidobacterium most often represented by B. infantis or (b) dysbiotic infants who were not stably colonized by Bifidobacterium. The bimodal nature of this distribution permitted the recognition of threshold values between the healthy and dysbiotic microbiomes, which signal dysbiosis if the value of the parameter is on the dysbiotic side of the threshold. Based on these observations, the methods of this invention provide for the detection of dysbiotic signals by determining the value of suitable parameters and comparing those values to the thresholds described herein. A list of suitable parameters is provided in Table 1.

TABLE 1
The comparison of Dysbiotic and healthy infants

| Parameter | Dysbiotic State | Healthy State |
|---|---|---|
| Bifidobacterium | Decreased | Increased |
| pH | Increased | Decreased |
| SCFA Levels | Decreased | Increased |
| lactate:acetate Ratio | Less than 0.55 | Greater than 0.55 |
| Organic Acid Content | Decreased | Increased |
| Pathogenic Bacteria | Increased | Decreased |
| Bifidobacterium: | Less than 1 | Greater than 1 |
| Enterobacteriaceae ratio | | or equal to 1 |
| LPS | Increased | Decreased |
| Antibiotic Resistance | Increased | Decreased |
| Genes | | |
| Cytokines | Increased | Decreased |
| MMO | Increased | Decreased |
| Colonization | Decreased: | Increased: |
| Resistance- | Jaccard stability | Jaccard stability |
| Microbiome Stability | index below 0.5 | index above 0.5 |

In some embodiments, a dysbiotic threshold is determined by an increase in cytokines; an increase in LPS; an increase in antibiotic resistance genes, increase in fecal pH above 5.85 and an increase in E. coli.

A simple, healthy infant microbiome can be described as the presence of greater than 10⁸ CFU/g stool of a single genus of bacteria (e.g., Bifidobacterium), more particularly, of a single subspecies or strain of bacteria (e.g., B. longum subsp. infantis). For example, up to 80% of the microbiome (relative abundance) can be dominated by the single bacterial species, particularly Bifidobacterium sp., or more particularly, by a single subspecies of a bacteria such as B. longum subsp. infantis. A simple microbiome can also be described as the presence of greater than 20%, preferably greater than 30%, more preferably greater than 40%, greater than 50%, greater than 60%, greater than 70%, greater than 75%, greater than 80%, or greater than 90% of a single genus of bacteria (e.g., Bifidobacterium), more particularly, of a single subspecies of bacteria (e.g., B. longum subsp. infantis) as measured by amplicoin metagnomic sequencing to establish relative abundance of the identified sequences or shotgun metabolomics (counts per million) and expressed as relative abundance (unitless) of the total microbiome. This population has features of ecological competitiveness, resilience, persistence, and stability over time, as long as MMO are present.

Monitoring Dysbiosis Via Fecal SCFA

Bifidobacterium are known to produce acetate and lactate. The total amount of these acids are higher in fecal samples having high Bifidobacterium compared to low Bifidobacterium samples—and not specifically a linear difference in pH. The level of organic acid and SCFA can be indicative of a healthy microbiome, and more specifically the preferred make-up of the distribution of organic acid and SCFA includes acetate and lactate. The SCFA can include formic, acetic, propionic, and butyric acids, and their salts. Preferably, the organic acid/SCFA include acetate and lactate which can make up at least 50% of the SCFA.

In some embodiments, a dysbiotic threshold is determined by a decrease in the lactate:acetate ratio away from 0.67 (2:3) towards 0.33 (1:3); in some embodiments the dysbiotic threshold is lactate:acetate less than 0.55; a decrease in organic acid content greater than 10 μmob or a decrease in total Bifidobacterium and/or B. infantis per gram of feces compared to a healthy infant. This embodiment is useful for monitoring the intestinal conditions in infants.

The level of bifidobacteria in an infant can be determined using a device that measures pH. The inventors have determined that pH levels in a stool sample correlate well to the levels of bifidobacteria in a microbiome (e.g., an infant microbiome). In a healthy infant microbiome, the inventors discovered that bifidobacteria will generate at least 30 μmol of titratable acidity in the form of organic acid and SCFA per gram of feces. In particular embodiments, the level of Bifidobacterium in a fecal sample is determined by measuring pH of a fecal sample, where pH above 5.85 may be interpreted to be from a human infant having low Bifidobacterium in the colon, and pH below 5.85 may be interpreted to be from a human infant having high Bifidobacterium in the colon.

A device that includes an indicator that indicates pH directly can be utilized with a fecal sample that may be deproteinated and/or filtered. Indicators such as, but not limited to, chlorophenol red (yellow to violet), transition from one color to another around pH 6.0 and may be used to visually discriminate a high bifidobacteria fecal sample from a low bifidobacteria fecal sample. A pH of 6.0 or below demonstrates that the sample has high levels of bifidobacteria. The device design may provide a window that gives a positive (high bifidobacteria) and negative (low bifidobacteria) sign to the user. Alternatively, users are provided a color card to match Bifidobacterium level to the color of the test result. In other embodiments, an optical reader, electrical probe or electrical sensor may be used to establish the ionic or colorimetric change associated with the pH differential.

There are various limitations on the usefulness of pH as a parameter for monitoring the microbiome. The fecal protein matrix may cause interference with pH measurements. Additionally, pH does not tell the full story because it only measures free hydrogen ions. In the infant gut, the acidity is also driven by the presence of short-chain fatty acids and in particular acetate and lactate that may not be disassociated. Titratable acidity is typically measured by determining the volume of 0.1 N NaOH required to change the pH to 8.2 using a pH electrode and calculating the concentration of titratable acidity within the test sample. In some embodiments, titratable acidity is tested using an alternative method that uses a fixed amount of NaOH and phenolphthalein to determine if the test sample has high titratable acidity (shifts pH below the threshold of 8.5) or low titratable acidity (does not shift pH below 8.5).

The titratable acidity of a solution is an approximation of the solution's total acidity. It includes both free hydrogen ions and also those still associated with the acid. In the present invention, the ratio of the NaOH and amount of fecal sample was determined to elicit a color change in the indicator at the cut-off between low and high abundance of Bifidobacterium in a sample set at 10⁸ CFU/gram of feces. The cut-off may also be expressed as CFU/μg DNA. The chemistry. High Bifidobacterium in this invention (at least 10⁸ CFU/gram of feces) can mean an amount of titratable acidity within 45-100 mg of feces that changes phenolphthalein (eg. 100 ul of 1% phenolphalein in 95% ethanol) from pink/fuchsia to colorless in the presence of a set amount of NaOH (eg. 63 μl 0.1 N NaOH mM in 1900 μl water=3.21 mM) having a pH of at least 11.4 @ 25 degrees Celsius before addition of phenolphthalein/ethanol mixture). In some embodiments the 5% alcohol may be made up of ethanol, methanol or other alchohols. The mixture of phenolphthalein and NaOH would be expected to be above 10.0 @ 25 degrees. Low Bifidobacterium in this invention (less than 10⁸ CFU/gram of feces) can mean an amount of titratable acidity within 45-100 mg of feces that cannot change phenolphthalein from pink/fuchsia in the presence of a set amount of NaOH.

In some cases, a dysbiotic threshold is determined as a short chain fatty acid concentration less than 50 μmol/g of feces and more preferably less than 35 μmol/g of feces (FIG. 13). The method can include the steps of: (a) obtaining a fecal sample from the mammal; (b) determining the level and composition of SCFA in the sample; (c) identifying a dysbiotic state in the mammal if the level of SCFA is too low or of skewed composition; (d) treating the dysbiotic mammal by: (i) administering a bacterial composition comprising bacteria capable of and/or activated for colonization of the colon; (ii) administering a food composition comprising MMO; or (iii) both (i) and (ii) added contemporaneously. This mode of the invention can provide a method of monitoring and/or maintaining the health of a mammal.

In particular embodiments, this invention provides a method of determining the level of Bifidobacterium in a fecal sample by measuring titratable acidity, the method comprising the steps of: (a) taking a predetermined amount of fecal sample, (b) mixing the fecal sample with a fixed amount of NaOH, (c) adding a 95% ethanol solution of 1% phenolphthalein to provide 0.048% phenolphthalein in the final mixture, and (d) monitoring the color of the resultant mixture, where mixtures that stay fuchsia or pink may be recognized to come from mammals having low bifidobacteria in their colon, and mixtures that change their color away from fuchsia/pink towards yellow/peach may be recognized as having come from mammals having high bifidobacteria levels in their colon. This embodiment is useful for monitoring the intestinal condition of a human infant.

A fecal sample can be added to a mixture that includes a fixed concentration of NaOH and an indicator. The fecal sample and NaOH can be in a ratio of 0.63-1.41 μmol of NaOH per gram of feces. In some embodiments, a device is designed to match the range of titratable acid in a certain amount of fecal sample (i.e., 45-100 mg) to a fixed concentration of NaOH or other base such that the indicator changes color to discriminate high vs low Bifidobacterium fecal samples. The device can include a basic solution selected from NaOH, KOH or any other appropriate base. A solution that includes 0.1M NaOH can also include deionized water to dilute to the appropriate range and/or ethanol or other suitable alcohols such as but not limited to methanol, propanol, and isopropanol. The device can include a reading window and a sampling device which can aide the user in providing a precise amount of the fecal material (e.g., 60 mg). The device may include a filter to remove the particulate matter. The fecal sample and indicator can be added contemporaneously into the device. In some embodiments, the indicator can be in a vessel into which the fecal sample and solution are introduced. The device can include a reading window to view the colorimetric reaction between the fecal sample, indicator and NaOH. If the device contains an indicator, such as phenolphthalein in ethanol whose color changes in the range of 8.2-8.7, the color of the resulting composition can indicate a threshold level of bifidobacteria in the sample.

In one embodiment, a kit according to this invention contains
Solution A: a 100 μl+/−10 μl of a 1% phenolphthalein 95% ethanol solution. This solution has a pH<8.5 and, thus, is colorless.
Solution B: 1963 μl+/−20 μl of a Sodium hydroxide solution (0.0321 N, pH>8.5, no indicator, colorless).

The reagents may be held in a single vessel/chambers or held in separate vessels/chambers until the kit is used. The kit is used when a fecal test sample is added to one or more of the solutions. In some embodiments, the test sample is added to B first and then A is added. In other embodiments, A and B are mixed to form before the test sample is added. They form Solution C (pH>8.5, fuchsia/pink).
Test sample 1: fecal sample from infant with low Bifidobacterium level;
Test sample 2: fecal sample from infant with high Bifidobacterium level.

If a given mass of test sample 1 is added to a known volume of solution B, the mixture will be of indeterminate color (poop colored; but not pink/fuchsia). If solution A is added in a known volume, then the solution will turn pink/fuchsia purple. If a given mass of test sample 2 is added to a known volume of solution B, the mixture will be of indeterminate color (poop colored; but not pink/fuchia). If solution A is added in a known volume, then the solution will not turn pink/fushia.

If Test sample 1 is added to solution C, the mixture will be fuchsia/pink. If Test sample 2 is added to solution C, the mixture will be stool color (yellow/peach).

In some embodiments, the vessel may contain one or more chambers, the vessel has a viewing window to observe the color change, and has a means of delivering a given mass of fecal sample to the vessel.

If the mixture of the fecal sample plus indicator phenolphthalein and NaOH has a pH of 8.5-8.7 or above, the fecal sample has a fecal pH of 5.85 or above and the sample would be described as low bifidobacteria. The pH of the composition is less than 8.5-8.7 the fecal sample would have had a pH of 5.85 or less and the sample would be described as high in bifidobacteria. Due to the discovery of the relationship between fecal pH and bifidobacteria levels, the indication of fecal pH and levels indicates the bifidobacteria levels in the sample (FIG. 11). Thus, a fecal sample with a low level of bifidobacteria will remain pink if phenolphthalein is the indicator. A fecal sample with a high level of bifidobacteria will turn the indicator from pink to yellow/peach. The working range of the test is from 10.2 for solution C down to 6.0 for high Bifidobacterium samples. Low Bifidobacterium samples will have a pink/fuchsia color and be in the range of 8.7 to 9.8. High Bifidobacterium samples will have a range of 8.6-to 6.0 and be anywhere from orange/peach-yellow to clear.

Bacterial Characteristics of the Dysbiotic Infant

The levels of pathogenic microorganisms in the gut of a healthy mammal may be reduced, as compared to a dysbiotic infant. In some embodiments, the pathogenic bacteria are reduced by greater than 10%, 15%, 25%, 50%, 75%, 80%, or 85% compared to dysbiotic infants. Pathogenic microorganisms include, but are not limited to: Clostridium, Escherichia, Enterobacter, Klebsiella, and Salmonella species, and their presence in the colon can be estimated by their presence in the feces of the mammal. Pathogenic bacterial overgrowth may include, but is not limited to, Enterobacteriaceae (e.g., one or more of Salmonella, E. coli, Klebsiella, or Cronobacter). Pathogenic bacterial overgrowth can also include bacteria of Clostridium difficile, Escherichia coli, and/or Enterococcus faecalis.

In some embodiments, the proportion of pathogenic bacteria is measured. A method of monitoring Enterobacteriaceae, more specifically E. coli, as a marker for antibiotic resistance. In other particular embodiments, a ratio of total Bifidobacterium:E. coli is used to determine dysbiosis in a human infant, where in a ratio less than 1 is indicative of dysbiosis, and a ratio of 1 or more is indicative of a healthy state. In some embodiments, the pathogenic bacteria are Enterobacteriaceae (e.g., one or more of Salmonella, E. coli, Klebsiella, or Cronobacter) and/or Clostridium difficile, Escherichia coli, and/or Enterococcus faecalis In some embodiments, a dysbiotic threshold is a ratio of Bifidobacterium:Enterobacteriaceae less than 1.

In some embodiments, LPS and/or pathogenic bacteria in the gut of a mammal are monitored. In some embodiments, a method of monitoring the levels of lipopolysaccharide (LPS) in the gut of a mammal is contemplated. By optimizing colon chemistry, reducing the capacity for LPS production, and/or reducing the levels of proinflammatory lipopolysaccharide (LPS) in the gut of a mammal, the level of LPS is reduced, as compared to a dysbiotic infant, by greater than 5%, 10%, 15%, 20%, 25%, 50%, 75%, 80%, or 85% by treatment with B. infantis. In some embodiments, the level of LPS is reduced, as compared to a dysbiotic infant, to below 0.7 endotoxin units (EU)/mL, below 0.65 EU/mL, 0.60 EU/mL, or below 0.55 EU/mL.

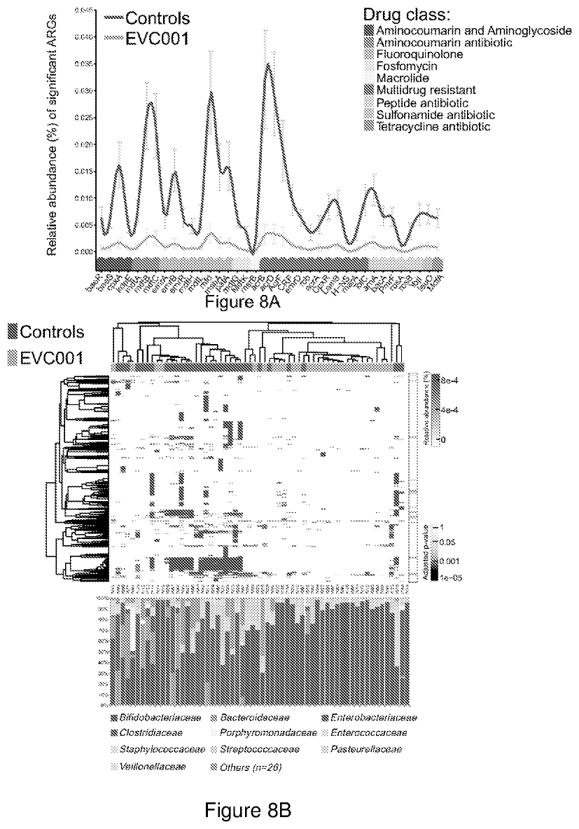

In some embodiments, a method of monitoring the antibiotic resistance gene load or the virulence gene is described. The method consists of monitoring a panel of one or more of the 38 ARGs genes identified in low Bifidobacterium samples (FIG. 8) or virulence genes. Shotgun metagenomics may be used to determine the ARG relative abundance in the microbiome. The expression of certain antibiotic resistant genes may be monitored in PCR based assys in isolated strains or a protein based assay to detect proteins contributing to an antibiotic resistant phenotype or a functional analysis of fecal isolates as measured by minimal inhibitory concentrations as exemplified in table 3. In other embodiments, antibiotic resistance gene load can be measured using the amount of Enterobacteriaceae per gram of feces. In a healthy microbiome, one or more genes of the antibiotic resistance gene load may be reduced by greater than 10%, 15%, 25%, 30%, 45%, 50%, 75% or 85% compared to the dysbiotic state. One or more genes of the virulence gene load may be reduced by greater than 10%, 15%, 25%, 30%, 45%, 50%, 75% or 85% compared to the dysbiotic state.

In some embodiments, the presence or absence of arabinose A and/or arabinose B genes can be used as a rapid test to discriminate B. longum from B. infantis. Colonization resistance is a critical function of the gut microbiome (Frese, 2017, mSphere 2:e00501-17. https://doi.org/10.1128/mSphere 0.00501-17). Stability of the gut microbiome is a measure of colonization resistance. Calculating similarities of the gut microbiome over time or to a baseline point provides a measure of stability at a given timepoint. In some embodiments, a Jaccard stability index (JSI) lower than 0.5 suggests dysbiosis, while a JSI higher than 0.5 suggests stability over time and absence of dysbiosis. The observed species index, Faith's phylogenetic diversity index [Faith DP. 1992. Conservation evaluation and phylogenetic diversity. Biol Consery 61:1-10.doi:10.1016/0006-3207(92)91201-3] and Shannon diversity index were used as metrics to compute alpha diversity. Weighted UniFrac distances were used as a beta diversity metric, in addition to the abundance-weighted Jaccard index, to calculate community compositional stability, congruent with previously described metrics of community stability Yassour et al. 2016. Natural history of the infant gut microbiome and impact of antibiotic treatment on bacterial strain diversity and stability. Sci Transl Med 8:343ra81. doi:10.1126/scitranslmed.aad0917; Faith J J et al. 2013. The long-term stability of the human gut microbiota. Science 341:1237439. doi:10.1126/science.1237439.

Markers of Inflammation

In other embodiments, a method of monitoring inflammation that may result from dysbiosis in the gut of a mammal comprises measuring the fecal levels of one or more of the following parameters: lipopolysaccharide (LPS); soluble toll-like Receptor-2 (sTLR2); soluble toll-like Receptor-4 (sTL4); soluble CD83; soluble CD14; and/or C-reactive protein (CRP) or fecal calprotectin. Fecal calprotectin is a marker of neutrophil and macrophage infiltration into inflamed intestinal tissue that can be detected in the stool. The above parameters can be used to assess the activity of groups of bacteria such as Enterobacteriaceae. This may be independent of the CFU per gram count of this group of bacteria. The method comprising taking a fecal sample to determine whether or not sample has greater than 10 ng/ml of sCD14 or sCD83. LPS may have a threshold of at least 2× the level found in the feces of infants having greater than 10⁸ CFU B. infantis/g feces. In some embodiments, a dysbiotic threshold for LPS may be considered a value above 5.36 log₁₀/ml. An intermediate value between 4.68 Log₁₀/ml and 5.36 Log₁₀/ml is considered inconclusive and requires other dysbiotic indicators to confirm dysbiosis.

In some embodiments, a fecal sample is assessed for multiple cytokines, receptors, and/or cell types related to inflammation. Inflammation is non-linear and multi-facetted. An algorithm can be used to determine if the cumulative effect of the different parameters exceed the threshold for dysbiosis (e.g., ranked importance of different markers, the number of markers above a dysbiotic threshold, the amount above the threshold to provide weighted values that indicate dysbiotic state or not). One or more of the following cytokines (pg/gram feces) have a threshold that is cytokine specific: IL-8 is greater than or equal to than 114; TNF-alpha greater than 6, INF-gamma greater than 51; IL-1beta is greater than 43; IL-22 is greater than 3; IL-2 is greater than 4; IL-5 is greater than 3; IL-6 is greater than 1; and IL-10 is greater than 1. In one embodiment the level above the threshold is considered specifically for IL-8, 11-10 and TNF-alpha; in other embodiments, IL-1B, INFgamma and TNF-alpha are considered together to determine presence or absence of dysbiosis. In yet other embodiments, the threshold for a particular cytokine or group of cytokines is determined based on the age of the infant.

In some embodiments, proinflammatory cytokines are monitored. Levels of proinflammatory cytokines including, but not limited to, IL-1 beta, IL-2, IL-5, IL-6, IL-8, IL-10, IL-13, IL-22, INF gamma and TNF-alpha, in a healthy infant are reduced relative to a dysbiotic infant, particularly by greater than 50%, greater than 60%, percent, greater than 70%, greater than 80%, greater than 90%, or greater than 95%. Reduction of the levels of proinflammatory cytokines including, but not limited to, IL-2, IL-5, IL-6, IL-8, IL-10, IL-13, and TNF-alpha, and/or increasing the levels of anti-inflammatory cytokines, in the gut of a mammal are consistent with removal of dysbiosis.

In some embodiments, residual fiber (e.g., MMO) can be a measure of dysbiosis: measure of total fiber of stool can be used to monitor or determine dysbiosis. In some embodiments, the threshold MMO level is at least 2×, at least 5× at least 10× higher than that of a healthy infant. In other embodiments, a fecal sample taken from a breast-fed infant is dysbiotic, if it has more than 10 mg total HMO/g feces, more than 20 mg total HMO/g feces, more than 40 total HMO/g feces.

### EXAMPLES

### Example 1: Trial with Breast-Fed Infants

This trial was designed to show the effect of probiotic supplementation with bifidobacteria in healthy term nursing infants compared to an unsupplemented group. A dry composition of lactose and activated Bifidobacterium long urn subsp. infantis was prepared starting with the cultivation of a purified isolate (Strain EVC001, Evolve Biosystems Inc., Davis, Calif., isolated from a human infant fecal sample EVC001 deposited under ATCC Accession No. PTA-125180) in the presence of BMO according to PCT/US2015/057226. The culture was harvested by centrifugation, freeze dried, and the concentrated powder preparation had an activity of about 300 Billion CFU/g. This concentrated powder was then diluted by blending with infant formula grade lactose to an activity level of about 30 Billion CFU/g. This composition then was loaded into individual sachets at about 0.625 g/sachet and provided to breast-fed infants starting on or about day 7 of life and then provided on a daily basis for the subsequent 21 days.

This was a 60-day study starting with infants' date of birth as Day 1. Before postnatal day 6, women and their infants (delivered either vaginally or by cesarean-section), were randomized into an unsupplemented lactation support group or a B. infantis supplementation plus lactation support group. Infant birthweight, birth length, gestational age at birth, and gender were not different between the supplemented and unsupplemented groups. Starting with Day 7 postnatal, and for 21 consecutive days thereafter, infants in the supplemented group were given a dose of at least 1.8×10¹⁰ CFU of B. infantis suspended in 5 mL of their mother's breastmilk, once daily. Because the provision of HMO via breastmilk was critical for supporting the colonization of B. infantis, all participants received breast feeding support at the hospital and at home and maintained exclusive breast feeding through the first 60 days of life. A subset of infants were followed out to 1 year of life.

Infant fecal samples were collected throughout the 60-day trial. Mothers collected their own fecal and breastmilk samples as well as fecal samples from their infants. They filled out weekly, biweekly and monthly health and diet questionnaires, as well as daily logs about their infant feeding and gastrointestinal tolerability (GI). Safety and tolerability was determined from maternal reports of infants' feeding, stooling frequency, and consistency (using a modified Amsterdam infant stool scale—watery, soft, formed, hard; Bekkali et al. 2009), as well as GI symptoms and health outcomes. Individual fecal samples were subjected to full microbiome analysis using Illumina sequencing based on 16S rDNA and qPCR with primers designed specifically for B. longum subsp. infantis strain.

Results

B. infantis was determined to be well-tolerated. Adverse events reported were events that would be expected in normal healthy term infants and were not different between groups. Reports specifically monitored blood in infant stool, infant body temperature and parental ratings of GI-related infant outcomes such as general irritability, upset feelings in response to spit-ups and discomfort in passing stool or gas, and flatulence. Furthermore, there were no differences reported in the use of antibiotics, gas-relieving medications, or parental report of infant colic, jaundice, number of illnesses, sick doctor visits and medical diagnoses of eczema.

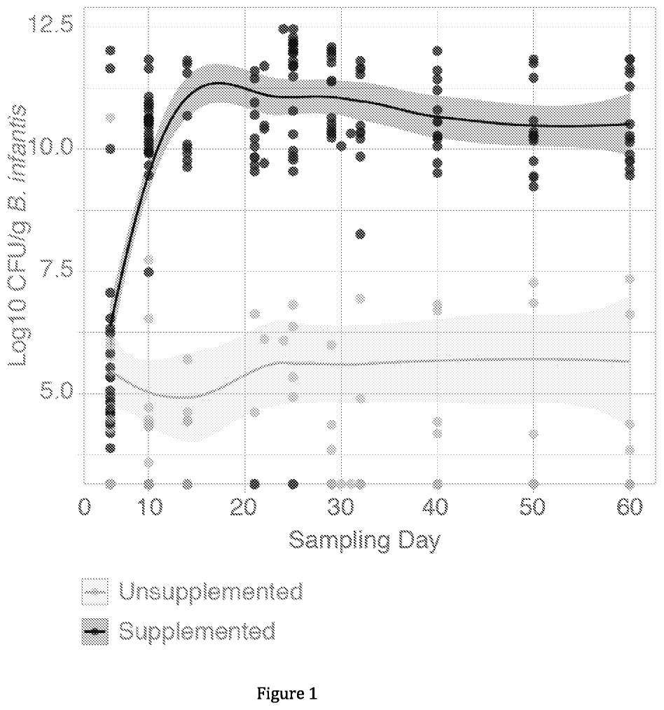

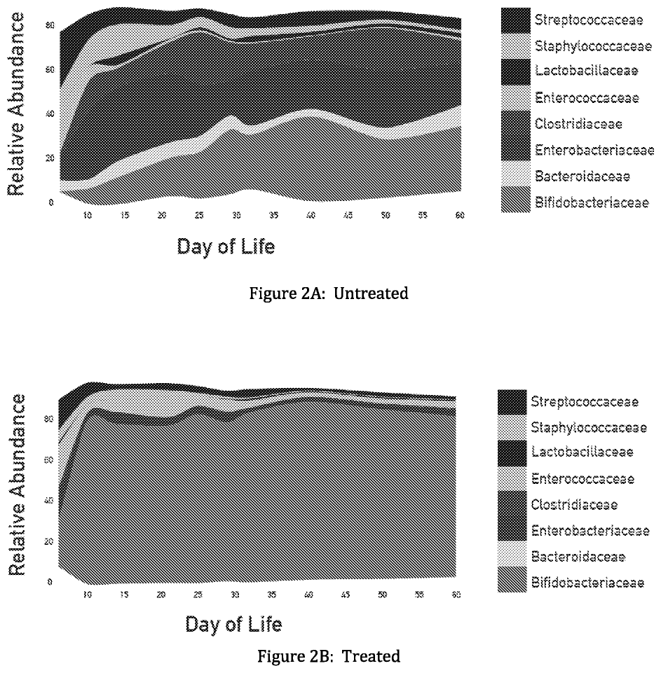

The B. infantis supplemented infants had a gut microbiome fully dominated (on average, greater than 70%) with B. longum subsp. infantis regardless of the birthing mode (vaginal or C-section). This dominance continued even after supplementation ended (Day 28) as long as the infant continued to consume breast milk, indicating that B. infantis was colonizing the infant gut to levels higher than 10¹⁰ CFU/g feces (FIG. 1). Furthermore, those infants that were colonized by the B. longum subsp. infantis also had much lower levels of proteobacteria and enterococci (including Clostridium and Escherichia species) (FIG. 2).

Unsupplemented infants (i.e., infants receiving the standard of care—lactation support but no supplementation of B. infantis) did not show B. infantis levels above 10⁶ CFU/g (i.e., the limit of detection) in their microbiome and there were significant differences in the microbiomes between C-section and vaginally delivered infants. Eighty percent (8 of 10) unsupplemented infants delivered by C-section had no detectable Bifidobacterium species and fifty-four percent (13 of 24) of the vaginally delivered infants had no detectable Bifidobacterium species by day 60. Further analysis of the thirteen unsupplemented infants that had some detectable bifidobacteria, found that the species were primarily B. longum subsp. longum, B. breve and B. pseudocatenulatum. No detectable B. longum subsp. infantis was found in any of the unsupplemented infants in the study.

The concentration of HMOs in infant feces was analyzed by liquid chromatography-mass spectrometry (LC-MS). The mean fecal HMO concentration in samples from B. infantis supplemented infants (4.75 mg/g) was 10-fold lower than in samples from unsupplemented infants (46.08 mg/g, P<0.001 by Tukey's multiple comparison test; FIG. 4).

When infant fecal samples were analyzed by LC-MS, B. infantis supplementation significantly increased fecal organic acids—particularly lactate and acetate. Other SCFAs (formate, propionate, butyrate, isovalerate, isobutyrate, and hexanoate) were in low abundance in the infant stool. Supplemented infants had significantly greater fecal organic acid concentrations than unsupplemented infants (126.55 μmol/g vs 52.02 μmol/g). The median lactate to acetate ratio of B. infantis-supplemented infants (0.73), was near the molar ratio of the “bifid shunt” (0.67), whereas low-bifidobacteria samples (the unsupplemented group) had a lactate to acetate ratio of 0.26 (P<0.0001, Mann-Whitney test).

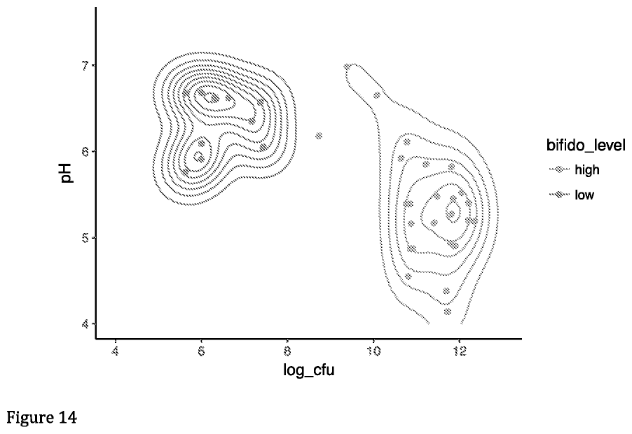

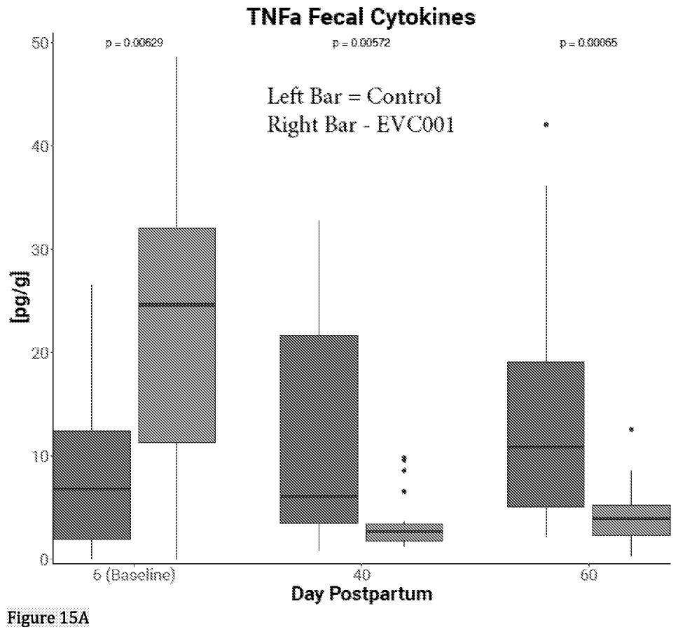

Monitoring pH in infant fecal samples showed a correlation between pH and the abundance of bifidobacteria in the sample. The mean fecal pH of the unsupplemented group was 5.97, while the feces from B. infantis-colonized infants had a significantly lower mean pH of 5.15 at day 21 postnatal (P<0.0001, Mann Whitney test). The pH of feces from that portion of unsupplemented infants who had no detectable bifidobacteria at all was 6.38, which was statistically higher than either of the other two groups (P<0.0001 Mann Whitney test). Overall, when compared across infants, absolute bifidobacteria populations in infant stools were negatively correlated with fecal pH (Spearman's p=−0.62, P<0.01) and demonstrated a bimodal distribution of fecal pH measurements that mirrored the abundance of bifidobacteria. Comparing weighted UniFrac distance matrixes, pH was a significant discriminator of sample community composition (Mantel Test, =0.32, P=0.002). FIG. 14 illustrates the bimodal distribution.

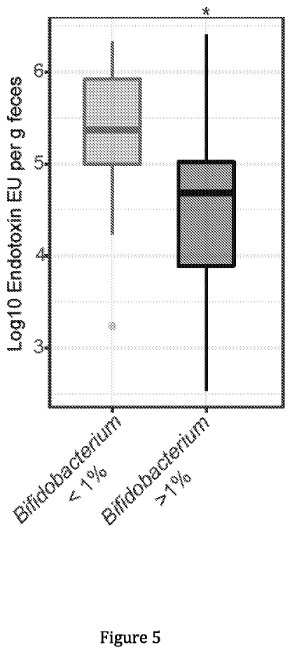

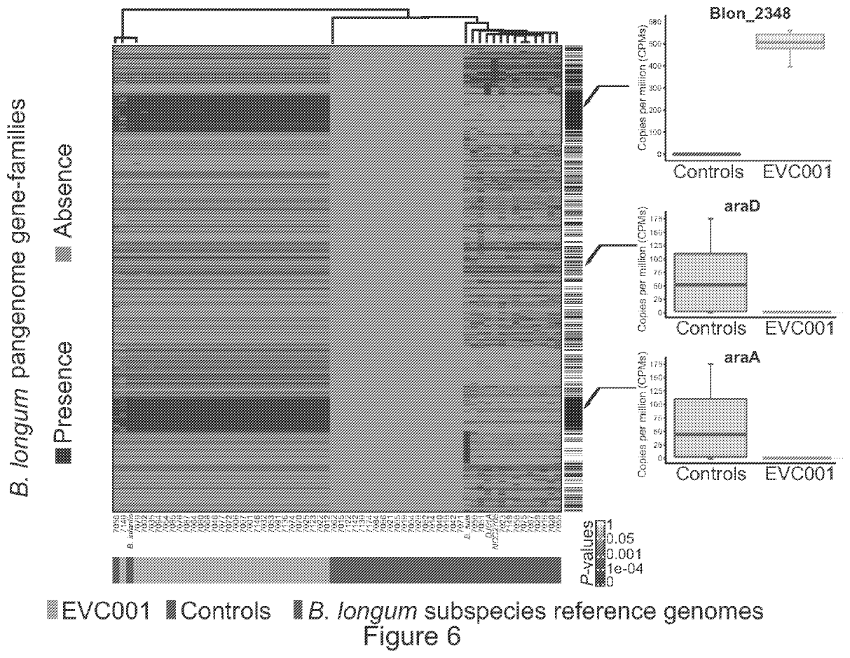

Measuring endotoxin (LPS) in the stool samples showed higher endotoxin in the unsupplemented infants (control) than in the supplemented infants (FIG. 5). The endotoxin load was nearly 4-fold lower in infants colonized at high levels with Bifidobacterium (>50% Bifidobacteriaceae) compared with endotoxin levels in infants with low levels of bifidobacteria, despite a high inter-individual variation (4.68 vs 5.36 Log₁₀ EU/mL, P=0.0252, Mann-Whitney U). Endotoxin was significantly correlated with Enterobacteriaceae relative abundance (P>0.0001, R=0.496), but not Bacteroidaceae, the second most abundant Gram-negative family found in the present study (P=0.2693), and endotoxin concentrations were inversely correlated with Bifidobacteriaceae abundance (P>0.001, R=−0.431). Thus, infants that had high levels of Bifidobacteriaceae colonization had lower endotoxin levels as compared to infants that did not have high levels of Bifidobacteriaceae colonization

This experiment demonstrates that non-dysbiotic infants can be identified as compared to dysbiotic infants by the following: (a) an increased in the lactate:acetate ratio to above 0.55 in the feces; (b) decreased inflammatory LPS by around 4× in the feces; (c) decreased pathogenic microbe levels in the feces; (d) decreased antibiotic resistance gene load by around 3× in the feces; (e) titratable acidity above 2 μmol/g feces, preferably above 5 μmol/g feces; (f) bifidobacteria levels of greater than 10⁷, preferably greater than 10^{8,} more preferably greater than 10⁹ in the feces; (g) B. infantis levels of greater than 10⁷, preferably greater than 10⁸, more preferably greater than 10⁹ in the feces; and/or (h) decreased HMO levels present in the feces of at least an order of magnitude, compared to dysbiotic infants. These parameter values may be expected to distinguish dysbiotic infants from non-dysbiotic infants across all mammals, not just human infants.

### Example 2: Measurement of Antibiotic Resistance Genes

Using the samples generated in Example 1, two different methods were first used to examine the fecal samples for antibiotic resistance gene (ARG) load present in the total microbiome of unsupplemented vs. B. infantis supplemented infants: 1) the Pfaffl method for relative abundance of a gene sequence (compared to 16S rRNA); and 2) a machine learning approach. To functionally classify the genes in fecal samples from unsupplemented or B. infantis supplemented groups, the 16S rRNA amplicon libraries generated were first organized into normalized, operational taxonomic unit (OTUs). PICRUSt, a publicly available bioinformatics freeware (picrust.github.io/picrust), was used to produce a table containing predicted gene classification of all the genes present. The genes were assigned using the Kyoto Encyclopedia of Genes and Genomes (KEGG) database (Kanehisa et al., 2000). Differences of predicted gene content in KEGG categories among samples were statistically analyzed using a Kruskal-Wallis one-way ANOVA with Bonferroni correction to adjust p-values.

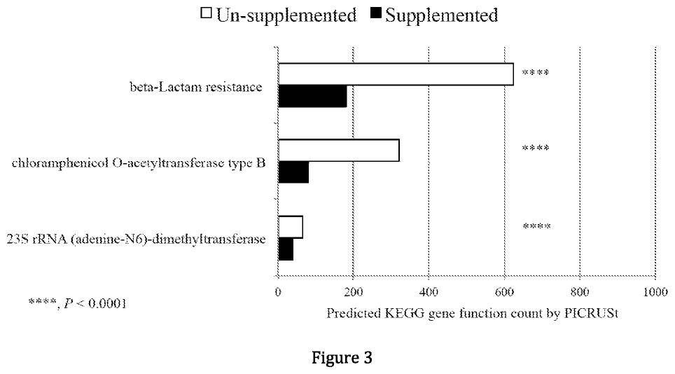

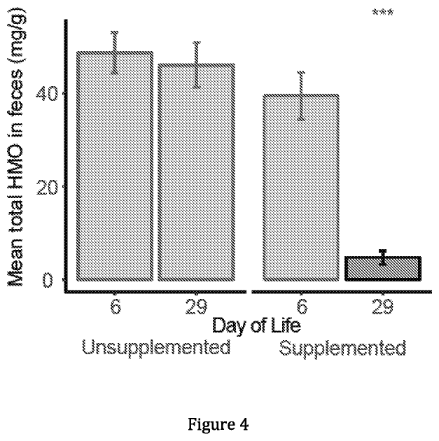

In B. infantis supplemented infants, erythromycin resistance genes (ermB) were reduced by about half in supplemented infants compared to unsupplemented infants using the Pfaffl Method for analyzing qPCR results (p=0.0258). Among the KEGG Orthologies identified, chloramphenicol O-acetyltransferase type B was significantly increased in the unsupplemented samples (p=5.50E-44; Bonferroni). Levels of the antibiotic resistance gene annotated as 23S rRNA (adenine-N6)-dimethyltransferase were significantly higher in the unsupplemented infants (p=1.32E-06; Bonferroni) than the supplemented infants. An entire group of antibiotic resistance genes were identified as beta-Lactam resistance genes and these genes were three times higher in the unsupplemented infants compared to the B. infantis supplemented infants (p=4.94e-56; Bonferroni) (FIG. 3).

Using shotgun metagenomic sequencing, the taxonomic as well as antibiotic resistance profiles were characterized within the gut microbiome of 60 healthy, term infants in Northern California (USA) at 21 days postnatal. Details of study design and subject characteristics have been reported previously (Smilowitz, J. T. et al. 2007. BMC pediatrics 17: 133). After quality filtering, Illumina sequencing led to a total of 1.6 billion paired end (PE) reads, of which about 3.6% were discarded as human contaminant, resulting in an average of 27 million PE reads per sample (Table 2). High-quality human-filtered reads were subjected to taxonomic profiling.

TABLE 2
Overview of recovered metagenomics sequencing results from supplemented
samples with EVC001 and unsupplemented controls.

| | | | Number of | Mean read | | Supplementation |
|---|---|---|---|---|---|---|
| Sample | Library ID in | Number of | human filtered | length | Number of | with B. infantis |
| ID | NCBI-SRA | QF reads | reads | (bp) | ARGs | EVC001 |
| 7020 | EBGC1A | 16,664,380 | 11,687,241 | 139.0 | 24.584 | No |
| 7022 | EBGC1B | 20,036,684 | 20,035,538 | 138.5 | 763 | Yes |
| 7006 | EBGC1C | 24,650,234 | 24,611,425 | 142.2 | 3.349 | Yes |
| 7064 | EBGC1D | 34,512,182 | 34,457,072 | 139.7 | 784 | Yes |
| 7042 | EBGC1E | 32,069,448 | 31,976,757 | 139.1 | 42.000 | No |
| 7085 | EBGC1F | 33,282,134 | 31,969,821 | 142.6 | 1.267 | Yes |
| 7071 | EBGC1G | 41,857,404 | 41,821,388 | 141.0 | 8.887 | No |
| 7018 | EBGC1H | 31,102,278 | 30,888,873 | 144.5 | 1.246 | No |
| 7053 | EBGC1I | 28,101,472 | 21,320,795 | 144.6 | 707 | Yes |
| 7046 | EBGC1J | 23,792,868 | 23,789,357 | 139.7 | 8.548 | Yes |
| 7029 | EBGC1K | 20,659,444 | 20,605,385 | 138.6 | 8.785 | No |
| 7040 | EBGC1L | 45,898,112 | 42,404,911 | 140.9 | 15.167 | No |
| 7002 | EBGC1M | 25,523,774 | 22,978,525 | 140.9 | 738 | Yes |
| 7070 | EBGC1N | 26,869,998 | 26,864,626 | 142.3 | 754 | Yes |
| 7055 | EBGC1O | 32,042,934 | 31,924,507 | 139.4 | 3.197 | No |
| 7025 | EBGC1P | 26,127,274 | 23,991,184 | 142.0 | 1.897 | Yes |
| 7014 | EBGC1Q | 20,842,834 | 15,924,419 | 139.0 | 22.159 | No |
| 7052 | EBGC1R | 20,538,332 | 20,105,645 | 138.8 | 46.173 | No |
| 7074 | EBGC1S | 32,128,230 | 32,127,264 | 141.3 | 6.301 | Yes |
| 7028 | EBGC1T | 29,018,642 | 28,901,182 | 141.2 | 41.122 | No |
| 7077 | EBGC2A | 38,565,654 | 38,524,801 | 141.8 | 1.068 | Yes |
| 7023 | EBGC2B | 29,636,288 | 28,626,060 | 138.8 | 4.104 | No |
| 7054 | EBGC2C | 31,704,534 | 31,703,776 | 137.0 | 9.46 | Yes |
| 7072 | EBGC2D | 28,452,700 | 28,389,141 | 143.1 | 2.473 | Yes |
| 7004 | EBGC2E | 21,966,018 | 21,892,596 | 140.9 | 25.796 | No |
| 7019 | EBGC2F | 22,137,906 | 21,102,023 | 137.1 | 50.744 | No |
| 7005 | EBGC2G | 24,739,036 | 23,824,316 | 137.5 | 9.539 | No |
| 7094 | EBGC2H | 23,002,228 | 22,456,171 | 136.4 | 6.009 | Yes |
| 7079 | EBGC2I | 29,178,840 | 29,166,934 | 139.2 | 55.263 | Yes |
| 7012 | EBGC2J | 24,605,428 | 24,554,918 | 139.9 | 643 | Yes |
| 7035 | EBGC2K | 22,606,816 | 22,593,152 | 142.5 | 3.228 | Yes |
| 7091 | EBGC2L | 19,907,888 | 19,866,462 | 140.3 | 437 | Yes |
| 7007 | EBGC2M | 19,783,706 | 19,219,848 | 139.1 | 373 | Yes |
| 7058 | EBGC2O | 19,895,006 | 17,875,331 | 139.8 | 2.664 | No |
| 7001 | EBGC2P | 19,391,072 | 19,383,270 | 138.6 | 328 | Yes |
| 7032 | EBGC2Q | 23,850,530 | 23,837,344 | 139.9 | 1.588 | Yes |
| 7021 | EBGC2R | 84,352,444 | 81,636,315 | 140.0 | 107.776 | No |
| 7075 | EBGC2S | 26,710,272 | 26,301,057 | 139.4 | 27.284 | No |
| 7067 | EBGC2T | 20,460,098 | 20,370,155 | 138.5 | 27.587 | No |
| 7086 | EBGC3A | 28,888,864 | 27,268,596 | 138.4 | 51.264 | No |
| 7084 | EBGC3B | 22,770,012 | 22,182,351 | 138.4 | 32.418 | No |
| 7068 | EBGC3C | 23,591,120 | 23,415,803 | 138.0 | 644 | Yes |
| 7080 | EBGC3D | 27,030,836 | 27,029,113 | 138.3 | 416 | Yes |
| 7149 | EBGC3E | 23,871,178 | 23,862,487 | 136.8 | 19.932 | No |
| 7076 | EBGC3F | 48,936,120 | 48,548,332 | 138.8 | 9.874 | Yes |
| 7146 | EBGC3G | 25,966,270 | 25,897,222 | 141.1 | 1.259 | Yes |
| 7140 | EBGC3H | 21,180,698 | 21,179,628 | 138.7 | 219 | Yes |
| 7056 | EBGC3I | 31,171,324 | 17,196,539 | 137.4 | 42.179 | No |
| 7174 | EBGC3J | 20,530,156 | 20,518,654 | 138.0 | 22.767 | No |
| 7130 | EBGC3K | 23,730,582 | 20,700,297 | 138.2 | 17.993 | No |
| 7136 | EBGC3L | 34,194,988 | 34,053,298 | 137.5 | 2.495 | Yes |
| 7142 | EBGC3M | 24,750,628 | 24,354,502 | 141.0 | 1.577 | No |
| 7087 | EBGC3N | 25,393,930 | 24,853,636 | 136.4 | 1.056 | Yes |
| 7016 | EBGC3O | 27,796,252 | 27,574,657 | 136.9 | 8.877 | No |
| 7122 | EBGC3P | 26,488,780 | 26,470,036 | 138.6 | 2.181 | No |
| 7050 | EBGC3Q | 29,278,320 | 29,179,951 | 137.3 | 12.681 | No |
| 7051 | EBGC3R | 23,552,354 | 23,542,902 | 134.9 | 2.028 | No |
| 7123 | EBGC3S | 31,897,560 | 31,584,466 | 137.3 | 1.801 | Yes |
| 7015 | EBGC3T | 24,645,758 | 24,512,684 | 136.2 | 1.300 | No |
| 7062 | EBGC3U | 23,476,062 | 23,422,279 | 140.8 | 36.751 | No |

A total of 202 bacterial species belonging to 76 genera, 43 families, 21 orders, 13 classes and 7 phyla were identified across samples. There were remarkable differences in the taxonomic distribution between the infants who were fed EVC001 and those who were not. Among infants fed EVC001, 10 bacterial genera made up 99% of the community, with the Bifidobacterium genus representing 88% of the total relative abundance of any identified genus (n=55) (P<0.0001; Kruskal-Wallis test) In the unsupplemented group, 68 genera were identified of which Bifidobacterium was present at only 38%, whereas other genera were increased, particularly Clostridium (P=0.01, Kruskal-Wallis test).

Within the Bifidobacterium genus, eight different species were identified. Bifidobacterium longum was the most abundant, representing 86% of the total identified bacterial species within the EVC001 supplemented infants and 19% within the unsupplemented controls (P<0.0001, Kruskal-Wallis test). Other detected bifidobacteria included B. breve and B. bifidum, which accounted for 9.4% and 7%, respectively, in the unsupplemented control infants and considerably less (1.4%, 0.4%, respectively) in the EVC001 supplemented group.

To discriminate the B. longum species at the subspecies level and determine the abundances of B. longum subsp. infantis and B. longum subsp. longum to specifically relate changes in microbiome composition to colonization by B. infantis, we performed a strain-level analysis within the B. longum species using the pangenome gene-families database provided by PanPhlan. This database includes genes from 38 strains of B. longum subspecies (e.g. B. longum subsp. longum, B. longum subsp. infantis, and B. longum subsp. suis). PanPhlan recovered an average of 98.8% of all genes present in Bifidobacterium longum subsp. infantis ATCC 1569724 from every sample in the EVC001-fed group, representing 2,449 pangenome gene families. In contrast, nineteen infants in the unsupplemented control group lacked any detectable reads mapping to B. longum subspecies genes in their metagenomes. The remaining unsupplemented samples (n=12) reported 43% coverage of B. infantis genes, while Bifidobacterium longum subsp. longum NCC2705 had the highest gene recovery (79%) across 1,708 pangenome gene families.

Samples and representative reference genomes were hierarchically clustered based on pair-wise similarities between strains calculated via Jaccard distance between gene family profiles (FIG. 6). The resulting heatmap showed that Bifidobacterium longum subsp. infantis was substantially more abundant than other Bifidobacterium longum subspecies in the supplemented group. On the righthand side of FIG. 6, individual gene ratios are enlarged to illustrate the density differences between B. infantis EVC001 and B. longum. Gene loci unique to the B. infantis reference genome and samples from B. infantis EVC001-fed infants revealed key genes including HMO clusters24. These genes were absent among 29 of 31 infants not fed with B. infantis EVC001, indicating that B. infantis was exceptionally rare (only 3% of infants) unless infants were fed B. infantis EVC001. Genes unique to B. longum subsp. longum that enable characteristic arabinose consumption, araD and araA, were significantly enriched among infants colonized by B. longum subsp. longum and rare among infants fed B. infantis EVC001. Together, this suggests that B. infantis EVC001 was the dominant B. longum subspecies among infants fed B. longum subsp. infantis EVC001.

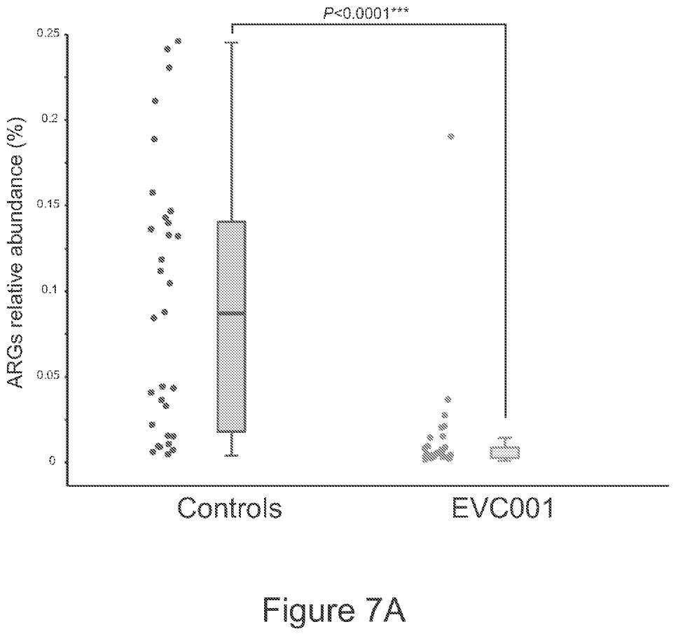

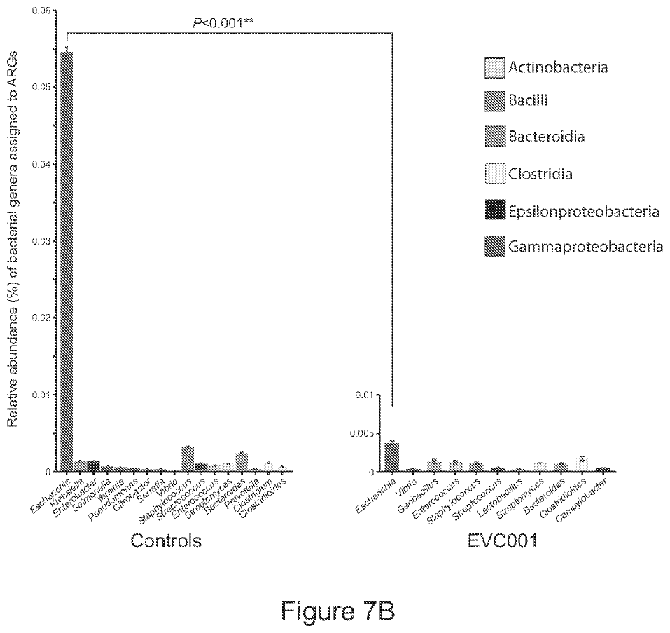

Supplementation with EVC001 was associated with reduction of ARG burden. We identified a total of 599,631 infant gut microbial genes from shotgun sequencing data in our study, of which 80,925 were unique to 29 infants who were fed B. infantis EVC001 and 313,683 microbial genes were unique to samples from 31 infants who were not fed B. infantis EVC001. Both groups shared a total of 205,023 microbial genes. Next, within the metagenomes we screened for ARGs using BLASTx type search against the curated Comprehensive Antibiotic Resistance Database (CARD). After quality filtering of BLAST results we identified a total of 652 ARGs. The EVC001-fed group reported an average of 0.01% of ARGs among total microbial genes (min=0,001%; max=0.18%; SEM=0.006%), with 285 different ARGs (FIG. 7, A) of which 33 were only found in the EVC001 group in very low percentages (<0.05%). Among infants not fed B. infantis EVC001, these ARGs accounted in average for 0.08% of the total metagenomic reads (min=0.004%; max=0.24%; SEM=0.01) with 612 different ARGs identified, of which 360 uniquely belonged to this group. Thus, infants fed EVC001 had, on average, 87.5% less ARGs in their microbiome (P<0.0001; Mann-Whitney test).

To compare the microbial taxonomic affiliation of ARGs, we assigned the 652 ARGs identified among the best BLAST hits to different taxa according to the NCBI taxonomy guidelines coupled with the Lowest Common Ancestor (LCA) method in MEGAN. A total of 41 bacterial genera were taxonomically assigned to the 652 ARGs, of which Escherichia, Staphylococcus, Bacteroides, Clostridioides were associated with the majority of the ARGs (68.9%; 5%; 4%; 2.6% respectively). Considering the taxonomic content within the resistome, metagenomes from infants not fed EVC001 had seventeen bacterial genera with a relative abundance>0.001%, with Escherichia-ARGs accounting for about 0.054% of the total metagenome (FIG. 7B). In the EVC001 group only 12 bacterial genera had a relative abundance of associated ARGs>0.001%. Escherichia was also the genus carrying the majority of ARGs but contributed significantly less to the overall metagenome (0.003%) of EVC001-fed infants compared to the unsupplemented controls (P=0.001, Kruskal-Wallis test; FIG. 7B).

EVC001 significantly decreased the abundance of key antibiotic resistant genes. Among the ARGs uniquely identified in the samples from infants not fed EVC001, three were present in a relative abundance greater than 0.1% and associated to the Clostridium genus. Specifically, we found tetA(P) and tetB(P), which are ARGs found on the same operon. tetA(P) is an inner membrane tetracycline efflux protein and tetB(P) is a ribosomal protection protein, both confer resistance to tetracycline25,26. We also found mprF uniquely in the samples from infants not fed EVC001, which activity negatively charges phosphatidylglycerol on the membrane surface and confers resistance to antibiotic cationic peptides that disrupt the cell membrane, including defensins27. After cross-sample normalization, 38 ARGs were significantly different between the two groups (P<0.01, Kruskal-Wallis test). All 38 ARGs were decreased in the EVC001 supplemented group. Notably, we did not identify any ARGs to be significantly increased in samples from the EVC001-fed group compared to the unfed group (P>0.05, Kruskal-Wallis test). Genes enriched in the metagenome of infants who were not fed EVC001 confer resistance to beta-lactams, fluoroquinolones and macrolides, and twelve genes confer resistance to multiple drug classes.

Hierarchical clustering of samples and genes using the complete-linkage method generated two main clusters of samples (FIG. 8B). The majority of the samples from the EVC001-fed infants clustered together within the lower-ARGs abundance panel. Row clustering by ARG resulted in two groups. The most abundant genes, which clustered together, were annotated as directly related to mechanisms of antimicrobial resistance. Particularly, proteins encoded by mdtB and mdtC form a heteromultimer complex resulting in a multidrug transporter28. AcrD is an aminoglycoside efflux pump and its expression is regulated by baeR and cpxAR, which were also identified among the significant ARGs and best characterized in E. coli. Moreover, we identified AcrB and TolC, which form the multidrug efflux complex AcrA-AcrB-TolC, which confers multidrug resistance. RosA and RosB were also significantly more abundant among infants not fed EVC001 and form an efflux pump/potassium antiporter system (RosAB) described in Yersinia. Finally, three genes belonging to the multidrug efflux system EmrA-EmrB-TolC first identified in E. coli were also significantly more abundant. In this complex, EmrB is the electrochemical-gradient powered transporter, while EmrA is the linker and TolC is the outer membrane channel. The complex confers resistance to fluoroquinolones antibiotics—nalidixic acid and thiolactomycin.

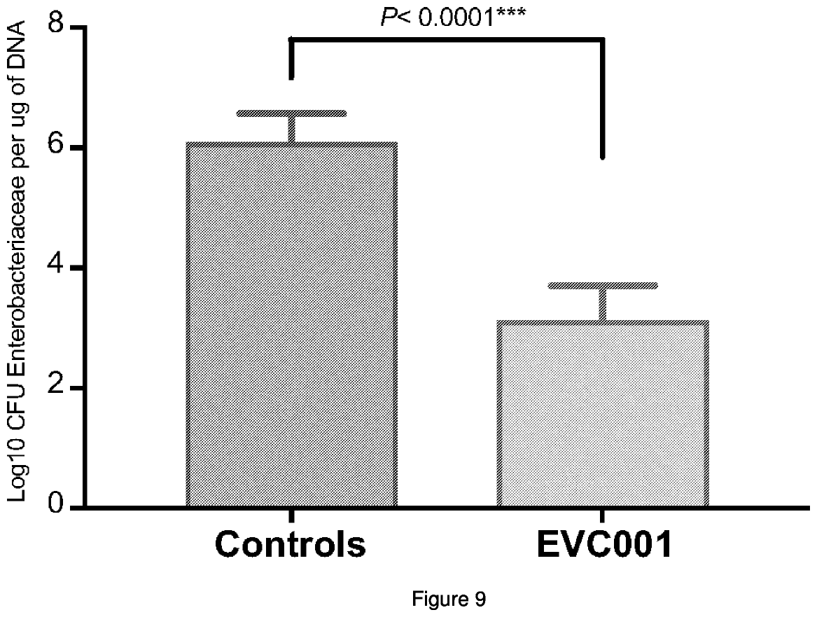

Overall, it appears the Enterobacteriaceae family is the main taxa contributing to the increased abundance of ARGs in the unsupplemented control infants. In fact, the majority (76%), of the significant ARGs were taxonomically assigned to bacteria belonging to the Enterobacteriaceae family (e.g. Escherichia coli) and its abundance is proportional to the presence of ARGs (R=0.58; P<0.00001; Pearson) (FIG. 8, B). Moreover, the absolute abundance (determined by qPCR) of Enterobacteriaceae is significantly reduced (P<0.0001) in EVC001-fed infants (FIG. 9).

Other ARGs reported multiple taxonomic assignments within the Proteobacteria phylum. According to NCBI's taxonomic assignment and the CARD database they could originate from any one of multiple, closely related species. These included the efflux pump acrD; the MdtG protein, which appears to be a member of the major facilitator superfamily of transporters, conferring resistance to fosfomycin and deoxycholate; BaeR a response regulator conferring multidrug resistance; and marA, a global activator protein overexpressed in the presence of different antibiotic classes.

PCR validation of in silico detected ARGs. In order to validate their presence in the fecal DNA, a PCR primer pair was designed for seven of the most abundant ARGs in the resistome of unsupplemented infants. Amplicons were obtained in at least half of the analyzed fecal samples, with the exception of the primers pairs targeting the mfd gene, which did not produce PCR products. Nucleotide sequence analysis of the generated amplicons revealed that the sequences corresponded to what was expected, as the vast majority had nucleotide identity of >70% to the open reading frame (ORF) of the target gene. Furthermore, nucleotide sequence analysis revealed high homology (85-99%) to genomic regions annotated to encode the expected functions in gut bacteria, and the predicted amino acid sequences contained highly conserved structural and functional domains in corresponding encoded proteins (Table 4).

Supplementation with EVC001 reduces total abundance as well as composition of ARGs. To compare the overall impact of EVC001 colonization on the diversity of antibiotic resistance genes, the alpha-diversity (e.g., number of unique ARGs observed) within each sample was compared using rarefaction curves. Notably, the diversity of ARGs was independent from the number of sequences per sample.

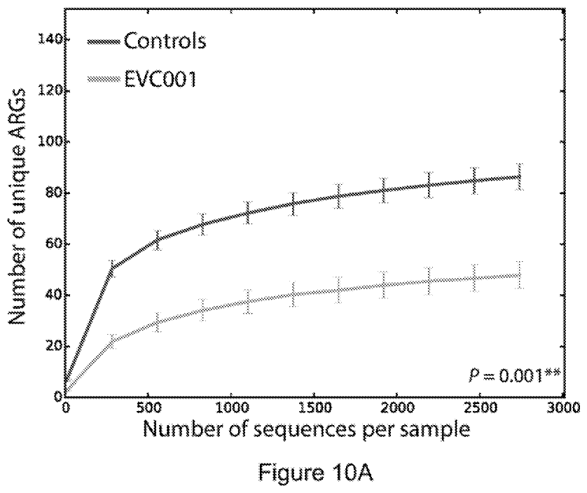

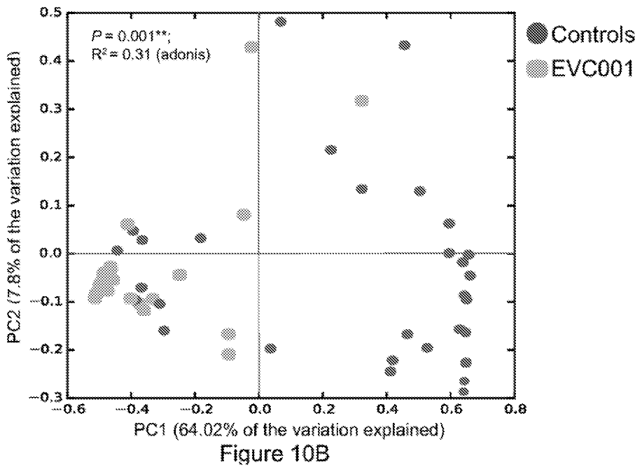

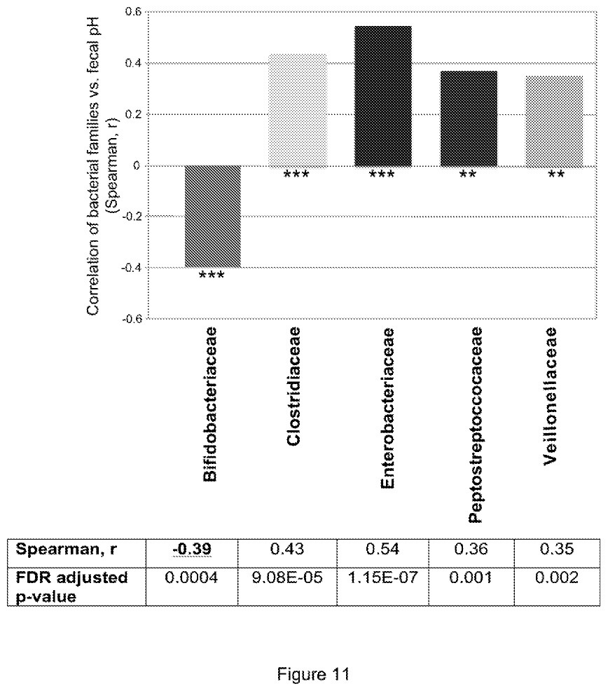

(FIG. 10A). Overall, the EVC001-fed infants had half as many unique ARGs as infants not fed EVC001 (P=0.001; T-test). FIG. 10B shows global resistome differences among samples and the effect-size of colonization by EVC001 on the overall diversity of the two study groups. A Bray-Curtis dissimilarity matrix transposed into principal coordinate analysis (PCoA) showed that samples from the EVC001-colonized group clustered closely together, compared to the control, which had a wider distribution (P=0.001, F-test). This indicates that samples from EVC001-colonized infants had a less abundant and less diverse resistome compared with the control group samples. Colonization with EVC001 contributed to a more than a 30% reduction in global AR diversity in the infant gut microbiome than in the gut of controls (R²=0.31, P=0.001, adonis).

To confirm the presence of full length, functional ARGs and the relationship of these ARGs to individual resistance phenotypes at the strain level, bacteria isolated from EMB agar were obtained from the fecal samples of four representative control infants. Whole-genome sequencing of twelve isolates was performed on a MinION sequencer and assembly led to an average coverage of 18× (min 5.4; max 40). Taxonomic identification was confirmed via BLASTN against the NCBI nucleotide database (https://www.ncbl.nlrn.nih.gov/nucleotide) showing three isolates classified as Raoultella planticola and the remaining nine as Escherichia coli. The CARD protein sequences collection was used as query against the twelve assembled isolates via TBLASTN. The presence of 38 significantly different ARGs identified via shotgun metagenomics was confirmed on the twelve genomes (average % identity >93), except for Streptomyces cinnamoneus EF-Tu, Yersinia enterocolitica rosB and Enterobacter cloacae rob. The latter genes are likely absent on the E. coli and R. planticola genomes and present on different species.

Whole genome sequencing and assembly of bacterial isolates. Approximately 100 mg of fecal sample from day 21 (subjects 7005, 7084, 7122 and 7174) were serially diluted onto EMB agar and incubated overnight at 37° C. Three colonies from each subject that were either dark in color and/or had a green metallic sheen were selected for subsequent analysis. Selected isolates were grown in 20 ml LB broth overnight at 37° C. Cultures were aliquoted into 1 ml aliquots, centrifuged at 10,000×g for 5 min and the supernatant was removed. Cell pellets were resuspended in DNA/RNA Shield solution provided in DNA/RNA Shield Microbe Lysis tubes (Zymo Research, Irvine Calif.) and transferred into lysis tubes. High-molecular weight genomic DNA was extracted using the Quick-DNA Fecal/Soil

TABLE 3
Minimum inhibitory concentrations (MIC) in μg/mL of antibiotics

| Isolate | Ampicillin | | Cefepime | | Cefotaxime | | Cefazolin | | Tetracycline | | Gentamicin | |
|---|---|---|---|---|---|---|---|---|---|---|---|---|
| 7005-5 | 128 | (R) | 16 | (R) | 32 | (R) | 64 | (R) | ND | | <4 | (S) |
| 7005-9 | 128 | (R) | 64 | (R) | 128 | (R) | 64 | (R) | ND | | <4 | (S) |
| 7005-11 | 64 | (R) | 512 | (R) | 512 | (R) | 64 | (R) | ND | | <4 | (S) |
| 7005-13 | 64 | (R) | 512 | (R) | 512 | (R) | 64 | (R) | ND | | <4 | (S) |
| 7084-1 | 512 | (R) | <4 | (S) | <4 | (S) | 32 | (R) | 16 | (R) | <4 | (S) |
| 7084-4 | 512 | (R) | <4 | (S) | <4 | (S) | 32 | (R) | 16 | (R) | <4 | (S) |
| 7084-5 | 512 | (R) | <4 | (S) | <4 | (S) | 32 | (R) | 16 | (R) | <4 | (S) |
| 7122-1 | 16 | (S) | <4 | (S) | <4 | (S) | 8 | (S) | 16 | (R) | <4 | (S) |
| 7122-2 | 16 | (S) | <4 | (S) | <4 | (S) | 8 | (S) | 8 | (S) | <4 | (S) |
| 7122-9 | 16 | (S) | <4 | (S) | <4 | (S) | 8 | (S) | 8 | (S) | <4 | (S) |
| 7174-2 | 512 | (R) | <4 | (S) | <4 | (S) | 64 | (R) | 64 | (R) | <4 | (S) |
| 7174-4 | 512 | (R) | <4 | (S) | <4 | (S) | 512 | (R) | 128 | (R) | <4 | (S) |
| 7174-5 | 512 | (R) | <4 | (S) | <4 | (S) | 512 | (R) | 64 | (R) | <4 | (S) |
| DH5α | 4 | (S) | <4 | (S) | <4 | (S) | <4 | (S) | <4 | (S) | <4 | (S) |

Resistant (R); Susceptible (S) according to breakpoints for clinical resistance (NCLSI, 2016).
ND not determined

Microbe Miniprep Kit (Zymo Research, Irvine, Calif.). DNA was extracted following the manufacturer's protocol with a mechanical lysis in a FastPrep96 (MP Biomedicals, Santa Ana, Calif.) for 15 sec at 1,800 rpm. gDNA integrity was assessed by gel electrophoresis using a high-molecular weight 1Kb Extension ladder (Invitrogen, Carlsbad, Calif.). Presence of gDNA band at 40kp and no shearing showed intact gDNA. gDNA was quantified using the Quant-iT™ dsDNA Assay Kit, high sensitivity (Invitrogen). gDNA purity was assessed using the Take3 microwell UV-Vis system (BioTek, Winooski, Vt.). Individually barcoded libraries were prepared for each isolate using 400 ng of high-molecular weight gDNA using Oxford Nanopore 1D Rapid Barcoding Kit (SQK-RBK004) (Oxford Nanopore Technologies, Oxford UK) according to manufacturer's protocol. Barcoded samples were pooled and a 1× HighPrep PCR bead clean-up (MagBio, Gaithersburg, Md.) of the fragmented and barcoded libraries prior to Rapid adapter ligation was included at the recommendation of Oxford Nanopore. The final 12-plexed pool was loaded on an R9.4 flow cell and run for 15 h. A secondary run was performed using the same protocol for the seven isolates whose initial coverage was below 6×. Reads were basecalled in real time using MinKnow (ONT, Oxford UK). Data for both runs were combined for subsequent processing. Basecalled reads were demultiplexed and adapters were trimmed using Porechop (version 0.2.3, https://github.com/rrwick/Porechop). Reads were assembled with Canu v1.5 {Koren, 2017} with default parameters. Assembled genomes were converted into local blast databases and the CARD database protein sequences were used as query against the assembled genomes using TBLASTN with min E-value set at 0.001. Genome assemblies were deposited on NCBI Gene Bank (https://www.ncbi.nlm.nih.gov/genbank/) with accession number PRJNA472982.

Minimal inhibitory concentrations. Minimal inhibitory concentrations (MICs) were determined according to Clinical and Laboratory Standards Institute guidelines for microdilution susceptibility testing {Wikler, 2006}. Strains grown in LB broth overnight were adjusted to 1×10⁶ CFU/ml and inoculated into Mueller-Hinton Broth containing binary combinations and one of twelve different pediatric-relevant antibiotics (ampicillin, tetracycline, cefataxime, cefazolin, cefepime) ranging from 0.5 to 512 μg/mL in 96-well polystyrene microtiter plates. Carbenicillin was added to growth media for transformed strains at a concentration of 100 μg/ml. The microtiter plates were incubated for 24 h at 37° C. The optical density (OD) of each well was measured at 600 nm using an automated microtiter plate reader (BIO-TEK, Synergy HT). The MIC corresponded to the lowest antibiotic concentration at which no growth was detected. All tests were performed in triplicate

The minimum inhibitory concentration (MIC) to ampicillin, cefepime, cefotaxime, cefazolin, tetracycline and gentamicin was determined for these isolates. With the exception of three isolates obtained from the same infant (7174), all of the isolates displayed resistance to ampicillin. Among multidrug-resistance isolates, resistance to ampicillin, cefazolin and tetracycline was the most common. No resistance to gentamicin was detected. To determine if the presence of an ARG could alter the sensitivity to antibiotics, the ORFs of the seven most abundant ARGs in the resistome of control infants compared with the EVC001-fed infants were synthesized and cloned into pRSETA vectors and each expressed in E. coli BL21 (DH3). No significant changes in antibiotics susceptibility were detected, suggesting that the expression of the individual genes alone was insufficient to confer a resistance phenotype.

TABLE 4
BLAST Global Alignment
BLASTx

| Conserved domain specific | | Conserved domain relevant | |
|---|---|---|---|
| hits [accession] | E-value | non-spefic hits [accession] | E-value |
| Multidrug efflux pump subunit | 8.88E−33 | Multidrug efflux system transporter | 6.07E−178 |
| AcrA [COG0845] | | AcrA [PRK15030] | |
| Multidrug efflux pump subunit | 1.95E−24 | Multidrug efflux system transporter | |
| AcrA [COG0845] | | AcrA [PRK15030] | |
| Multidrug efflux pump subunit | 1.53E−16 | Multidrug efflux system transporter | 3.11E−112 |
| AcrA [COG0845] | | AcrA [PRK15030] | |
| Multidrug efflux pump subunit | 2.29E−31 | Multidrug efflux system transporter | 1.10E−161 |
| AcrA [COG0845] | | AcrA [PRK15030] | |
| Signal transduction histidine | 3.17E−08 | two-component sensor protein, | 7.39E−160 |
| kinase [COG0642] | | cpxA [PRK09470] | |
| TonB-dependent Receptor | 402E−06 | Outer membrane cobalamin receptor | 1.14E−04 |
| Plug Domain [pfam07715] | | protein [COG4206] | |
| n/a | | DNA-binding transcriptional activator | 0.00E+00 |
| | | MarA [PRK11511] | |
| SeIR domain, methionine sulfoxide | 1.43E−45 | Peptide methionine sulfoxide reductase | 3.26E−53 |
| reductase, pfam01641 | | MsrB [COG0229] | |
| ABC-type transport system | 3.31e−106 | | |
| [COG3839] | | | |

BLASTn

| | % | | | | |
|---|---|---|---|---|---|
| Closest homologue [organism] | identity | E value | Score | | GenBank |
| efflux transporter periplasmic adaptor | 96 | 0 | 1823 | bits(987) | ASK00454.1 |
| subunit [Citrobacter freundii] | | | | | |
| efflux transporter periplasmic adaptor | 98 | 0 | 1879 | bits(1017) | ASK00454.1 |
| subunit [Citrobacter freundii] | | | | | |
| MexE family multidrug efflux RND | 98 | 0 | 1943 | bits(1052) | ATM90194.1 |
| transporter periplasmic adaptor subunit | | | | | |
| [Klebsiella aerogenes] | | | | | |
| MexE family multidrug efflux RND | 95 | 0 | 1746 | bits(945) | ATR17048.1 |
| transporter periplasmic adaptor subunit | | | | | |
| [Klebsiella pneumoniae] | | | | | |
| Two component system sensor histidine | 98 | 0 | | | ATM14937.1 |
| kinase CpxA [Raoultella planticola] | | | | | |
| Outer membrane receptor protein | 99 | 0 | 645 | bits(349) | APW88916.1 |
| [Klebsiella variicola] | | | | | |
| MDR efflux pump AcrAB transcriptional | 94 | 2E−133 | 486 | bits(263) | ATM18484.1 |
| activator MarA [Raoultella planticola] | | | | | |
| peptide-methionine (R)-S-oxide reductase | 99 | 4E−179 | 638 | bits(345) | ATM16796.1 |
| ABC transporter | 94 | 0 | 1674 | bits(906) | ATP54276.1 |
| [Collinsella aerofaciens] | | | | | |

### Example 3: A Method of Establishing a Visible Threshold for the Titratable Acidity in a Set Amount of Feces to Discriminate a Low Vs High Level of Bifidobacterium in a Fecal Sample

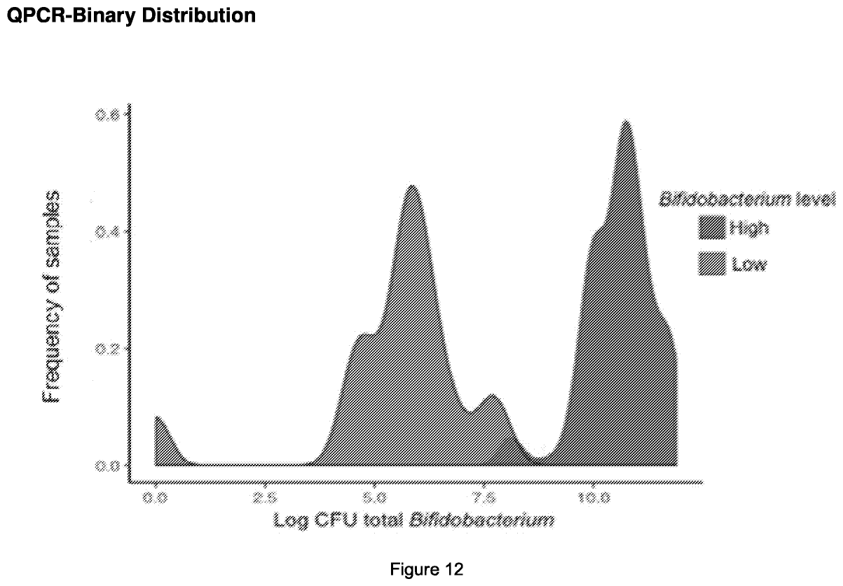

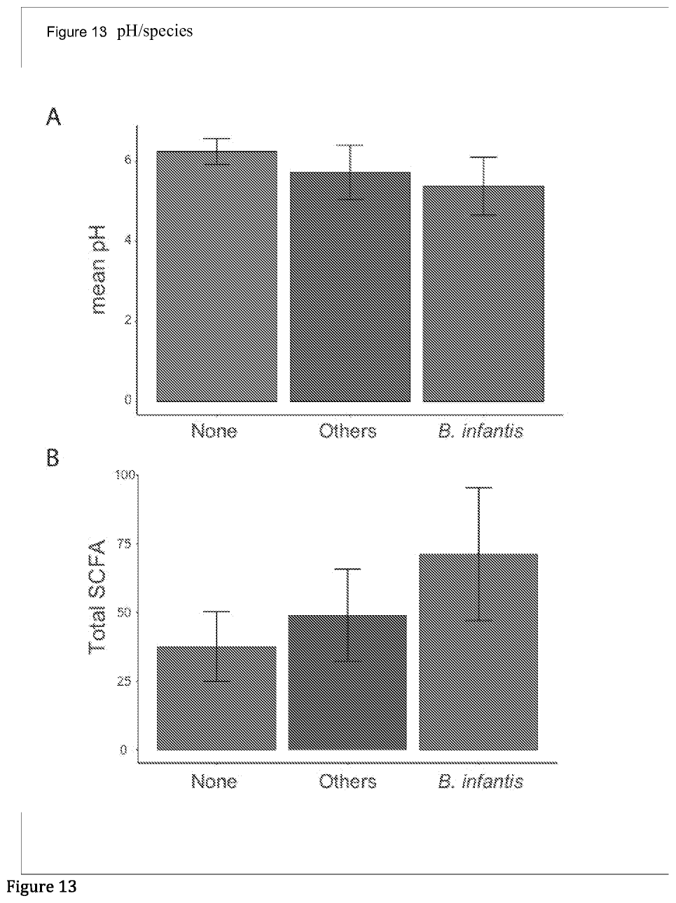

A target pH of 5.85 was determined as a threshold to separate the vast majority of fecal samples from control infants in the clinical study described in Example 1 into those with high Bifidobacterium levels from those with low Bifidobacterium levels (FIG. 14). A bimodal distribution of Bifidobacterium populations was found in samples of infant feces from Example 1 as shown in FIG. 12. A high level of Bifidobacterium in a sample was described as total Bifidobacterium greater than 10⁸ CFU/gram of feces, whereas a low level of Bifidobacterium in a sample was described as having less than 10⁸ CFU/gram (FIG. 12). It was also determined that the titratable acid (organic acids and short-chain fatty acids) in a fecal sample from infants without Bifidobacterium was different to fecal samples from EVC001 colonized infants and that this is especially the case for acetate and lactate (FIG. 13B). The total acetate and lactate was highest in infant fecal samples with high levels of B. infantis, compared to other samples from infants colonized by other Bifidobacterium, or to samples from infants with no Bifidobacterium (FIG. 13B). Titratable acidity was found to be a better measure to discriminate samples with low levels of Bifidobacterium from samples with high levels of Bifidobacterium, compared to pH alone (FIG. 13A).

Phenolphthalein is a pH indicator that is colorless below pH 8.5 and fuchsia/pink above pH 8.5. NaOH was used to shift the pH cut-off from 5.85 to 8.5-8.7 in the test system, such that the phenolphthalein color change discriminated a low level of Bifidobacterium (pink/fuchsia) from a high level of Bifidobacterium (yellow/peach). In order to develop the test, the pKa for acetate and lactate were used to calculate the amount of hydrogen ions expected in approximately 60 mg of feces from infants with high and low levels of Bifidobacterium after determining the absolute amount of acetate and lactate in those samples (μmol/gram feces).

In this experiment, Solution A (1% Phenolphthalein in ethanol solution, colorless) and solution B (Sodium hydroxide solution (pH>8.5, colorless) were premixed. The resulting solution C was pink/fuchsia before any fecal sample was added, indicating that the solution contained an excess of hydroxide (—OH) ions and the pH was greater than pH 8.5. The starting pH of solution C was 10.0-10.2.

Specifically, the amount of NaOH added in the test was calculated such that the H⁺ from a fecal sample with a low level of Bifidobacterium would be insufficient to quench the added NaOH. This excess of hydroxide ions would keep the pH of the solution above pH 8.5, and the solution, including the phenolphthalein indicator, would remain pink/fuchsia. However, the H⁺ ions in a sample with high levels of Bifidobacterium would exceed the concentration of —OH ions added, and the buffering effect will prevent the pH from exceeding pH 8.5. Thus, in the presence of the phenolphthalein indicator, the indicator would turn colorless if the sample in question came from an infant colonized in high levels by Bifidobacterium. The resulting sample is yellow/peach due to the color of the feces. The test results in a highly discriminative binary color separation between samples with low Bifidobacterium levels and samples with high Bifidobacterium levels, because the concentration of NaOH used in the test is fixed, and the final pH is dependent on the total amount of acidity in the starting fecal sample.

We determined that 0.63 μl of 0.1 N NaOH was required in a final volume of 2.063 mls to adjust the pH of the system such that the color change was able to separate a low level of Bifidobacterium from a high level of Bifidobacterium. The final NaOH concentration within the test was 0.0324 mmol/ml (63 μl of 0.1 N NaOH in a total volume of 2.063 mls). This means that there was a total of 0.0668 mmol in the test. The test was determined to be accurate between 45-100 mg of feces; therefore a ratio of 0.67-1.49 mmol/gram of feces is useful for this invention.

Stool samples collected from the trial described in example 1 were analyzed to determine if a sample had a low level of Bifidobacterium or a high level of Bifidobacterium based on whether or not a set amount of fecal sample (45-100 mg) contained enough titratable acidity to change 1% phenolphthalein (100 μl) in a mixture of 63 μl 0.1 N NaOH/1900 μl water after being shaken. The color of the test mixture was observed and recorded in Table 5. The same samples were analyzed by qPCR to classify them as low or high levels of total Bifidobacterium based on the bimodal distribution (FIG. 12). For example, the resultant mixture from the fecal sample of an unsupplemented infant was fuchsia or pink, indicating that the titratable acidity was below the threshold to change the phenolphthalein and that this infant has a low level of Bifidobacterium. In contrast, the resultant mixture from the B. infantis-supplemented infant was yellow/peach indicating that the fecal sample had enough titratable acidity to neutralize the base and bring the pH below the point where phenolphthalein changes to colorless and that the infant microbiome contains high bifidobacteria. A total of 129 samples were analyzed and the sensitivity of the test based on fecal titratable acidity was 94.52%; the specificity was 94.64%; the positive predictive value (PPV) was 95.83%; and negative predictive value (NPV) was 92.98%.

TABLE 5
The number of times the titratable acidity was able to predict the
level of Bifidobacteirum in a fecal sample.

| Test Result | Low | High | |
|---|---|---|---|
| using | Bifidobacterium < | Bifidobacterium | |
| phenolphthalein | 10E+08 CFU/g | CFU/g > 10E+08 | |
| Yellow (#) | 3 | 69 | 72 |
| Pink/Fuchsia (#) | 53 | 4 | 57 |
| Total Samples | 56 | 73 | 129 |

Acetic acid has a density of 1.050 g/ml, a molarity of 17.4 g/mol and a pKa of 4.75. Lactic acid has a density of 1.206 g/ml, a molarity of 11.3 g/mol and a pKa of 3.86.

It was determined that the amount of [H+] ions at pH 5.85 was 1.413 E-06 and a low Bifidobacterium sample that registered a pH of 5.97 would have [1.072E-06] H+ ions. A high Bifidobacterium sample would have [2.4E-06] H+ ions. The amount of NaOH added (0.63 μl 0.1 NaOH, pH 10) shifts the pH such that amount of H+ ions in the low Bifidobacterium sample are insufficient to drop the pH below 8.5 and that the high Bifidobacterium sample has sufficient [H+] ions to drop the pH below 8.5.

The principle demonstrated here can be applied to other threshold values of the invention, and one skilled in the art will recognize that the amounts are scalable, and the cut-offs can be shifted as required to apply the invention to different conditions, such as different species of mammals, different ages, different fecal sample quantities, etc.

### Example 4: Determination of Intestinal Inflammatory Activity to Assess Status of Dysbiosis

As part of the IMPRINT clinical study described in Example 1, healthy, exclusively breastfed infants were randomly selected to receive B. infantis EVC001 daily for 21 days or receive lactation support alone. Both groups were followed up to Day 60 postnatal (Smilowitz J T et al. 2017. BMC Pediatrics. 17:133. doi:10.1186/s12887-017-0886-9; Frese et al, 2017. mSphere 2:e00501-17. https://doi.org/10.1128/mSphere 0.00501-17]. Stool samples from this study were randomly selected from 20 infants who were fed EVC001 and 20 infants that received lactation support alone at Days 6 (baseline), 40 and 60, and analyzed for multiple proinflammatory cytokines, including IL-1beta, IL-2, IL-5, IL-6, IL-8, IL-22, INF-gamma, and TNF-alpha using the U-PLEX Biomarker Group 1 (human) 9-plex multiplex kit, Meso Scale Discoveries (Rockville, Md.) as shown previously Houser et al, 2018. Calprotectin levels were quantified using ELISA (Immundiagnostik, Germany).

Cytokines were measured according to Manufacturer's instructions using the Meso Scale Discovery (MSD) multi-spot assay system with U-plex or ultra-sensitive kits. Calibration curves from recombinant cytokine standards were prepared with fivefold dilution steps in supplied diluent. Standards were measured in duplicate, samples were measured twice, and blank values were subtracted from all readings. All assays were carried out directly in a 96-well plate at room temperature and protected from light. Briefly, wells were washed with 150 μl PBS containing 0.05% Tween 20, then standards and samples, or blank were added in a final volume of 25 and incubated at room temperature for 2 hours with continuous shaking. Wells were washed three times with 150 μl PBS containing 0.05% Tween 20. Detection antibodies (25 μl/well) were added to wells for a further 1 hour incubation at room temperature with continuous shaking. Wells were washed three times with PBS containing 0.05% Tween 20, then read buffer was added to each well. The plate was then read on a Sector Imager 2400. MSD Discovery Workbench analysis software with 4-parameter logistic curve-fitting was used for data analysis.

TABLE 5
Levels of fecal cytokines in fecal samples from Control (unsupplemented infants −
B. infantis) and EVC001 (infants supplemented + B. infantis EVC001) over 60 days.
All values are reported as pg cytokine per gram of feces. Average (Avg); standard deviation
(sdev); Interquartile Range (IQR); first quartile (first quant); third quartile (third quant).

| Cytokine | Treatment | Day | avg | sdev | IQR | first_quant | median | third_quant | min | max |
|---|---|---|---|---|---|---|---|---|---|---|
| IFNg | Control | 6 | 0 | 0 | 0 | 0 | 0 | 0 | 0 | 1 |
| IFNg | Control | 40 | 103 | 171 | 65 | 21 | 31 | 86 | 0 | 629 |
| IFNg | Control | 60 | 313 | 380 | 421 | 53 | 119 | 474 | 16 | 1476 |
| IFNg | EVC001 | 6 | 1 | 2 | 0 | 0 | 0 | 1 | 0 | 10 |
| IFNg | EVC001 | 40 | 15 | 18 | 12 | 5 | 13 | 18 | 0 | 86 |
| IFNg | EVC001 | 60 | 38 | 43 | 42 | 9 | 27 | 51 | 0 | 203 |
| IL10 | Control | 6 | 12 | 53 | 0 | 0 | 0 | 0 | 0 | 236 |
| IL10 | Control | 40 | 1 | 2 | 1 | 1 | 1 | 1 | 0 | 11 |
| IL10 | Control | 60 | 1 | 1 | 1 | 0 | 1 | 1 | 0 | 3 |
| IL10 | EVC001 | 6 | 7 | 6 | 9 | 1 | 7 | 10 | 0 | 18 |
| IL10 | EVC001 | 40 | 1 | 0 | 0 | 0 | 1 | 1 | 0 | 2 |
| IL10 | EVC001 | 60 | 1 | 1 | 0 | 0 | 0 | 1 | 0 | 4 |
| IL1B | Control | 6 | 620 | 1431 | 159 | 66 | 133 | 225 | 20 | 5750 |
| IL1B | Control | 40 | 3793 | 9241 | 2047 | 51 | 237 | 2098 | 10 | 35845 |
| IL1B | Control | 60 | 1961 | 2774 | 3147 | 46 | 725 | 3192 | 5 | 9748 |
| IL1B | EVC001 | 6 | 86 | 169 | 70 | 17 | 39 | 87 | 2 | 794 |
| IL1B | EVC001 | 40 | 42 | 75 | 31 | 13 | 20 | 43 | 5 | 374 |
| IL1B | EVC001 | 60 | 60 | 193 | 27 | 8 | 13 | 35 | 2 | 943 |
| IL2 | Control | 6 | 115 | 502 | 0 | 0 | 0 | 0 | 0 | 2248 |
| IL2 | Control | 40 | 5 | 8 | 2 | 2 | 3 | 4 | 1 | 36 |
| IL2 | Control | 60 | 11 | 13 | 17 | 2 | 3 | 19 | 0 | 44 |
| IL2 | EVC001 | 6 | 47 | 41 | 50 | 13 | 43 | 63 | 0 | 146 |
| IL2 | EVC001 | 40 | 5 | 4 | 4 | 2 | 3 | 6 | 0 | 20 |
| IL2 | EVC001 | 60 | 3 | 2 | 3 | 1 | 3 | 4 | 0 | 8 |
| IL22 | Control | 6 | 4 | 3 | 3 | 3 | 4 | 5 | 0 | 12 |
| IL22 | Control | 40 | 16 | 37 | 6 | 3 | 5 | 10 | 2 | 168 |
| IL22 | Control | 60 | 18 | 17 | 25 | 5 | 7 | 30 | 2 | 56 |
| IL22 | EVC001 | 6 | 4 | 2 | 2 | 3 | 4 | 5 | 1 | 10 |
| IL22 | EVC001 | 40 | 3 | 2 | 2 | 1 | 2 | 3 | 1 | 9 |
| IL22 | EVC001 | 60 | 4 | 3 | 2 | 3 | 4 | 5 | 1 | 14 |
| IL5 | Control | 6 | 23 | 89 | 6 | 0 | 0 | 6 | 0 | 400 |
| IL5 | Control | 40 | 2 | 2 | 1 | 1 | 2 | 3 | 1 | 10 |
| IL5 | Control | 60 | 6 | 7 | 6 | 2 | 3 | 8 | 1 | 32 |
| IL5 | EVC001 | 6 | 15 | 12 | 17 | 5 | 15 | 22 | 0 | 41 |
| IL5 | EVC001 | 40 | 2 | 1 | 1 | 1 | 1 | 2 | 0 | 6 |
| IL5 | EVC001 | 60 | 2 | 2 | 2 | 1 | 2 | 3 | 0 | 7 |
| IL6 | Control | 6 | 1 | 4 | 0 | 0 | 0 | 0 | 0 | 18 |
| IL6 | Control | 40 | 4 | 10 | 2 | 1 | 1 | 2 | 0 | 46 |
| IL6 | Control | 60 | 8 | 9 | 13 | 1 | 3 | 14 | 0 | 31 |
| IL6 | EVC001 | 6 | 8 | 9 | 9 | 1 | 7 | 10 | 0 | 31 |
| IL6 | EVC001 | 40 | 1 | 1 | 1 | 0 | 0 | 1 | 0 | 4 |
| IL6 | EVC001 | 60 | 1 | 1 | 1 | 0 | 1 | 1 | 0 | 5 |
| IL8 | Control | 6 | 878 | 1938 | 517 | 5 | 30 | 521 | 0 | 7316 |
| IL8 | Control | 40 | 4014 | 10286 | 1158 | 115 | 413 | 1273 | 51 | 43779 |
| IL8 | Control | 60 | 3050 | 3945 | 5510 | 130 | 470 | 5639 | 1 | 11455 |
| IL8 | EVC001 | 6 | 265 | 407 | 223 | 20 | 48 | 242 | 8 | 1153 |
| IL8 | EVC001 | 40 | 98 | 88 | 72 | 42 | 82 | 114 | 5 | 356 |
| IL8 | EVC001 | 60 | 272 | 737 | 120 | 35 | 70 | 154 | 5 | 3582 |
| TNFa | Control | 6 | 37 | 134 | 10 | 2 | 7 | 12 | 0 | 606 |
| TNFa | Control | 40 | 28 | 67 | 18 | 3 | 6 | 22 | 1 | 302 |
| TNFa | Control | 60 | 16 | 15 | 14 | 5 | 11 | 19 | 2 | 52 |
| TNFa | EVC001 | 6 | 26 | 21 | 21 | 11 | 25 | 32 | 0 | 79 |
| TNFa | EVC001 | 40 | 3 | 3 | 2 | 2 | 3 | 3 | 1 | 10 |
| TNFa | EVC001 | 60 | 4 | 3 | 3 | 2 | 4 | 5 | 0 | 13 |

TABLE 6
Levels of fecal cytokines in fecal samples at Day 6 of Life (before
treatment) compared to percentage of Bifidobacterium in the
total microbiome as measured by 16 s genomic sequencing.

| Sample | Percent_Bif | IFNg | IL10 | IL1B | IL2 | IL22 | IL5 | IL6 | IL8 | TNTa |
|---|---|---|---|---|---|---|---|---|---|---|
| 7085 | 85% | 1 | 18 | 33 | 100 | 5 | 28 | 10 | 22 | 49 |
| 7025 | 83% | 1 | 8 | 9 | 54 | 4 | 5 | 10 | 28 | 23 |
| 7007 | 64% | 0 | 6 | 96 | 79 | 1 | 12 | 9 | 13 | 25 |
| 7058 | 63% | 0 | 0 | 70 | 0 | 3 | 11 | 0 | 447 | 8 |
| 7029 | 53% | 0 | 0 | 140 | 0 | 5 | 0 | 0 | 7 | 3 |
| 7091 | 29% | 0 | 17 | 88 | 15 | 3 | 17 | 1 | 1094 | 11 |
| 7064 | 28% | 0 | 10 | 15 | 44 | 6 | 22 | 7 | 106 | 29 |
| 7123 | 25% | 0 | 0 | 87 | 0 | 2 | 4 | 0 | 1153 | 2 |
| 7140 | 13% | 0 | 1 | 794 | 13 | 4 | 15 | 4 | 242 | 27 |
| 7018 | 4% | 0 | 0 | 224 | 16 | 2 | 5 | 1 | 847 | 7 |
| 7045 | 0% | 0 | 10 | 48 | 43 | 4 | 16 | 7 | 538 | 29 |
| 7142 | 0% | 0 | 0 | 106 | 0 | 5 | 0 | 0 | 0 | 0 |
| 7028 | 0% | 0 | 0 | 49 | 0 | 0 | 0 | 0 | 34 | 0 |
| 7052 | 0% | 0 | 0 | 23 | 0 | 5 | 0 | 0 | 266 | 0 |
| 7084 | 0% | 0 | 236 | 5750 | 2248 | 12 | 400 | 18 | 7316 | 606 |
| 7075 | 0% | 0 | 0 | 421 | 0 | 5 | 10 | 0 | 298 | 4 |
| 7004 | 0% | 0 | 0 | 180 | 0 | 6 | 6 | 0 | 2398 | 6 |
| 7046 | 0% | 1 | 10 | 14 | 101 | 4 | 22 | 21 | 17 | 38 |
| 7005 | 0% | 0 | 0 | 229 | 8 | 5 | 6 | 0 | 59 | 8 |
| 7089 | 0% | 0 | 9 | 8 | 34 | 2 | 10 | 7 | 167 | 19 |
| 7019 | 0% | 0 | 0 | 112 | 0 | 2 | 0 | 0 | 4 | 7 |
| 7040 | 0% | 1 | 0 | 1038 | 0 | 3 | 0 | 0 | 26 | 9 |
| 7014 | 0% | 0 | 0 | 74 | 0 | 7 | 2 | 0 | 746 | 14 |
| 7072 | 0% | 0 | 9 | 97 | 52 | 2 | 30 | 10 | 16 | 32 |
| 7062 | 0% | 0 | 0 | 25 | 0 | 1 | 0 | 0 | 0 | 0 |
| 7012 | 0% | 0 | 0 | 195 | 0 | 3 | 0 | 0 | 8 | 2 |
| 7015 | 0% | 0 | 0 | 154 | 0 | 3 | 0 | 0 | 5 | 0 |
| 7002 | 0% | 1 | 15 | 27 | 107 | 5 | 33 | 26 | 20 | 69 |
| 7042 | 0% | 0 | 0 | 127 | 19 | 4 | 12 | 0 | 6 | 27 |
| 7001 | 0% | 10 | 18 | 17 | 146 | 10 | 41 | 31 | 33 | 79 |
| 7006 | 0% | 0 | 6 | 20 | 39 | 5 | 17 | 10 | 50 | 23 |
| 7086 | 0% | 0 | 0 | 20 | 0 | 3 | 0 | 0 | 2 | 16 |
| 7020 | 0% | 0 | 0 | 3469 | 0 | 7 | 0 | 4 | 5085 | 12 |
| 7087 | 0% | 1 | 4 | 18 | 63 | 8 | 24 | 11 | 48 | 41 |
| 7136 | 0% | 1 | 7 | 70 | 62 | 5 | 8 | 3 | 20 | 30 |
| 7021 | 0% | 0 | 0 | 139 | 0 | 2 | 0 | 0 | 19 | 14 |
| 7094 | 0% | 0 | 3 | 83 | 23 | 4 | 7 | 0 | 980 | 0 |

TABLE 7
Levels of fecal cytokines in fecal samples at Day 40 of Life compared to Percentage
of Bifidobacterium in the total microbiome as measured by 16 s genome sequencing

| Sample | Binned_Day | Percent_Bif | IFNq | IL10 | IL1B | IL2 | IL22 | IL5 | IL6 | IL8 | TNTa |
|---|---|---|---|---|---|---|---|---|---|---|---|
| 7136 | 40 | 98% | 26 | 1 | 12 | 2 | 3 | 3 | 0 | 89 | 2 |
| 7025 | 40 | 97% | 3 | 1 | 16 | 3 | 4 | 1 | 1 | 117 | 3 |
| 7087 | 40 | 97% | 18 | 0 | 18 | 4 | 1 | 2 | 0 | 136 | 1 |
| 7002 | 40 | 95% | 17 | 1 | 45 | 3 | 2 | 2 | 0 | 109 | 4 |
| 7140 | 40 | 95% | 15 | 1 | 8 | 3 | 3 | 2 | 1 | 13 | 2 |
| 7058 | 40 | 94% | 0 | 1 | 10 | 2 | 2 | 1 | 0 | 51 | 2 |
| 7085 | 40 | 94% | 0 | 1 | 37 | 7 | 1 | 1 | 0 | 11 | 1 |
| 7094 | 40 | 91% | 14 | 1 | 39 | 6 | 4 | 2 | 0 | 64 | 3 |
| 7042 | 40 | 91% | 15 | 1 | 15 | 2 | 3 | 2 | 0 | 69 | 1 |
| 7064 | 40 | 91% | 27 | 1 | 29 | 8 | 2 | 1 | 1 | 356 | 3 |
| 7007 | 40 | 91% | 9 | 1 | 51 | 3 | 1 | 1 | 0 | 259 | 2 |
| 7001 | 40 | 90% | 86 | 2 | 28 | 9 | 1 | 1 | 0 | 155 | 7 |
| 7089 | 40 | 90% | 7 | 1 | 70 | 3 | 3 | 2 | 0 | 254 | 3 |
| 7040 | 40 | 89% | 31 | 2 | 612 | 11 | 17 | 2 | 5 | 9710 | 33 |
| 7123 | 40 | 88% | 32 | 1 | 8 | 2 | 3 | 2 | 1 | 38 | 3 |
| 7142 | 40 | 88% | 37 | 1 | 219 | 4 | 6 | 3 | 1 | 500 | 7 |
| 7091 | 40 | 86% | 25 | 1 | 54 | 6 | 2 | 2 | 0 | 5 | 3 |
| 7006 | 40 | 84% | 13 | 0 | 41 | 1 | 1 | 1 | 1 | 111 | 2 |
| 7072 | 40 | 82% | 5 | 0 | 16 | 1 | 1 | 1 | 0 | 85 | 2 |
| 7029 | 40 | 70% | 16 | 1 | 36 | 1 | 2 | 2 | 1 | 69 | 4 |
| 7045 | 40 | 67% | 13 | 1 | 374 | 20 | 1 | 1 | 0 | 11 | 1 |
| 7046 | 40 | 66% | 6 | 1 | 47 | 2 | 2 | 1 | 0 | 106 | 4 |
| 7019 | 40 | 57% | 48 | 1 | 292 | 3 | 3 | 2 | 1 | 472 | 6 |
| 7018 | 40 | 56% | 21 | 0 | 59 | 2 | 3 | 1 | 1 | 477 | 5 |
| 7020 | 40 | 55% | 38 | 1 | 2331 | 4 | 9 | 2 | 2 | 2314 | 22 |
| 7015 | 40 | 44% | 29 | 1 | 255 | 3 | 6 | 1 | 1 | 477 | 6 |
| 7004 | 40 | 1% | 62 | 1 | 56 | 1 | 8 | 1 | 0 | 184 | 5 |
| 7075 | 40 | 1% | 31 | 1 | 634 | 3 | 11 | 2 | 1 | 926 | 10 |
| 7084 | 40 | 0% | 629 | 11 | 35845 | 36 | 168 | 10 | 46 | 43779 | 302 |
| 7014 | 40 | 0% | 512 | 1 | 23899 | 10 | 43 | 3 | 17 | 17549 | 83 |
| 7052 | 40 | 0% | 213 | 0 | 4558 | 1 | 4 | 3 | 0 | 107 | 22 |
| 7028 | 40 | 0% | 21 | 1 | 106 | 1 | 4 | 1 | 1 | 355 | 3 |
| 7005 | 40 | 0% | 26 | 1 | 75 | 4 | 5 | 2 | 1 | 317 | 5 |
| 7062 | 40 | 0% | 160 | 1 | 4782 | 4 | 6 | 2 | 2 | 224 | 9 |
| 7021 | 40 | 0% | 21 | 1 | 31 | 2 | 5 | 2 | 1 | 81 | 3 |
| 7086 | 40 | 0% | 159 | 2 | 2020 | 6 | 16 | 6 | 2 | 2501 | 26 |

TABLE 8
Levels of fecal cytokines in fecal samples at Day 60 of Life compared to percentage
of Bifidobacterium in the total microbiome as measured by 16 s genome sequencing

| Sample | Percent_Bif | IFNg | IL10 | IL1B | IL2 | IL22 | IL5 | IL6 | IL8 | TNTa |
|---|---|---|---|---|---|---|---|---|---|---|
| 7094 | 98% | 51 | 1 | 9 | 5 | 4 | 3 | 0 | 33 | 6 |
| 7140 | 96% | 26 | 0 | 6 | 2 | 4 | 3 | 1 | 62 | 6 |
| 7085 | 95% | 42 | 1 | 39 | 1 | 5 | 2 | 0 | 130 | 4 |
| 7025 | 95% | 63 | 1 | 35 | 4 | 6 | 1 | 2 | 235 | 6 |
| 7123 | 92% | 6 | 0 | 35 | 1 | 3 | 1 | 1 | 5 | 4 |
| 7136 | 92% | 32 | 0 | 2 | 3 | 2 | 2 | 1 | 28 | 5 |
| 7001 | 92% | 42 | 1 | 15 | 3 | 3 | 1 | 1 | 126 | 2 |
| 7007 | 92% | 203 | 0 | 66 | 7 | 14 | 5 | 5 | 3582 | 9 |
| 7064 | 91% | 11 | 0 | 42 | 0 | 4 | 1 | 1 | 277 | 0 |
| 7089 | 91% | 88 | 0 | 943 | 3 | 6 | 2 | 2 | 691 | 6 |
| 7029 | 85% | 19 | 1 | 5 | 0 | 2 | 1 | 1 | 30 | 2 |
| 7087 | 84% | 56 | 1 | 30 | 8 | 5 | 3 | 0 | 178 | 4 |
| 7002 | 82% | 34 | 0 | 7 | 4 | 3 | 2 | 0 | 48 | 4 |
| 7058 | 82% | 16 | 0 | 8 | 1 | 4 | 1 | 1 | 45 | 3 |
| 7006 | 81% | 3 | 0 | 7 | 0 | 1 | 0 | 0 | 27 | 2 |
| 7042 | 71% | 65 | 1 | 72 | 3 | 5 | 2 | 1 | 208 | 7 |
| 7045 | 70% | 51 | 1 | 12 | 4 | 4 | 3 | 0 | 56 | 5 |
| 7072 | 69% | 64 | 0 | 41 | 3 | 2 | 2 | 0 | 70 | 4 |
| 7091 | 68% | 23 | 0 | 15 | 2 | 4 | 1 | 1 | 105 | 2 |
| 7020 | 51% | 214 | 0 | 822 | 7 | 11 | 4 | 4 | 1477 | 17 |
| 7062 | 48% | 473 | 1 | 3113 | 18 | 25 | 8 | 14 | 3998 | 19 |
| 7015 | 44% | 78 | 0 | 353 | 3 | 6 | 3 | 1 | 331 | 10 |
| 7019 | 43% | 119 | 1 | 965 | 7 | 18 | 5 | 4 | 1536 | 11 |
| 7142 | 5% | 50 | 0 | 20 | 1 | 6 | 2 | 1 | 470 | 3 |
| 7004 | 1% | 56 | 1 | 106 | 2 | 5 | 1 | 3 | 282 | 6 |
| 7028 | 0% | 58 | 0 | 89 | 2 | 4 | 2 | 1 | 328 | 6 |
| 7075 | 0% | 475 | 1 | 2049 | 20 | 34 | 7 | 14 | 4986 | 14 |
| 7084 | 0% | 706 | 1 | 7227 | 30 | 56 | 32 | 31 | 11455 | 36 |
| 7021 | 0% | 16 | 0 | 16 | 1 | 3 | 3 | 0 | 1 | 3 |
| 7052 | 0% | 460 | 1 | 4417 | 17 | 27 | 8 | 11 | 6293 | 32 |
| 7014 | 0% | 255 | 0 | 9748 | 2 | 7 | 1 | 1 | 47 | 52 |
| 7005 | 0% | 634 | 1 | 4227 | 28 | 42 | 13 | 25 | 9407 | 42 |
| 7040 | 0% | 1476 | 3 | 725 | 44 | 34 | 8 | 14 | 6615 | 13 |
| 7086 | 0% | 761 | 1 | 3272 | 25 | 49 | 8 | 18 | 10383 | 19 |

Infants treated with B. infantis EVC001 (Table 5) or those with at least 53% of their microbiome being Bifidobacteriaceae (Table 7, Table 8) indicating that the colon of these infants is in a far calmer state with respect to inflammatory responses and would be considered healthy. Infants with lower percentages of Bifidobacteriaceae would be considered dysbiotic at levels less than 50% in this study. Newborn infants at Day 6 have a different pattern of cytokines than Day 40 or 60 in either group (Table 6).

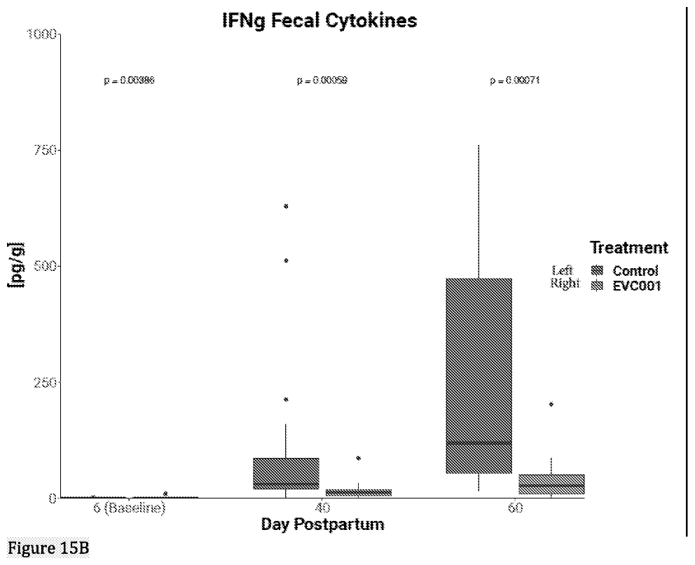

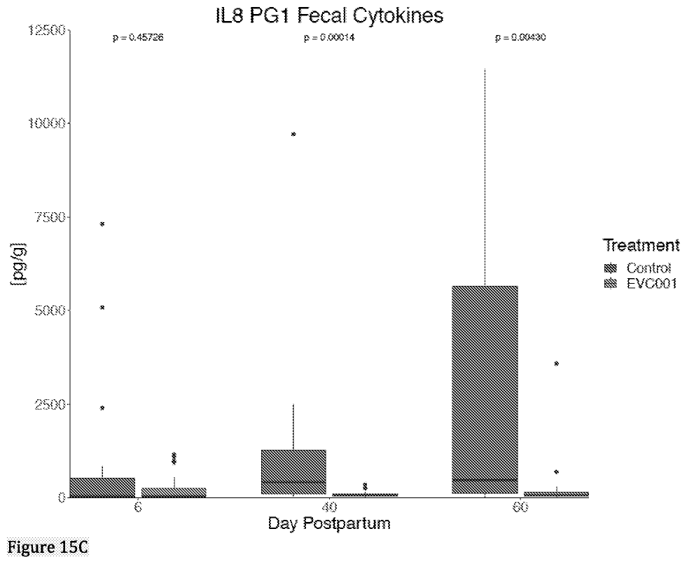

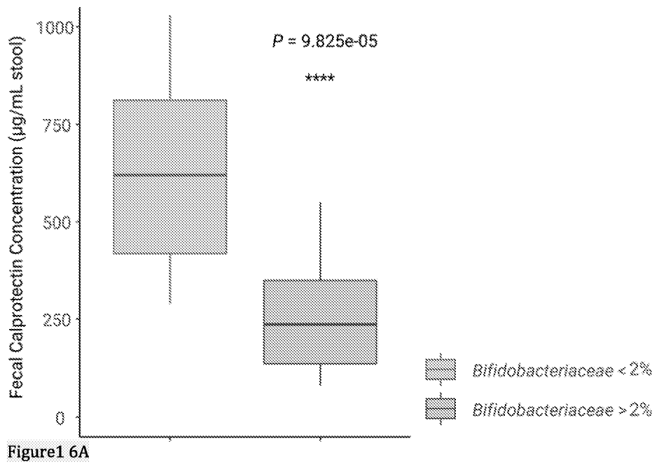

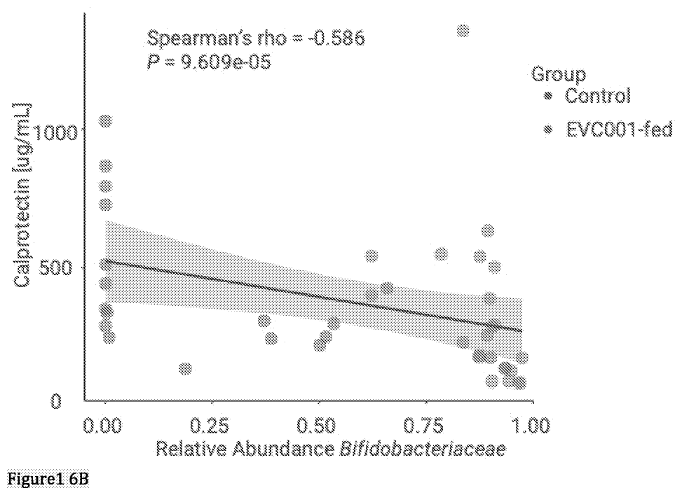

A typical immune response to pathogens involves the rapid activation of proinflammatory cytokines (e.g., IL-8 and TNF-a) that serve to initiate host defense against microbial invasion (FIGS. 15A and 15B respectively). Since excess inflammation can give rise to systemic disturbances harmful to the host, the immune system has evolved parallel anti-inflammatory mechanisms that serve to curb the production of proinflammatory molecules to limit tissue damage. Interleukin 10 (IL-10), a molecule that can limit host immune response to pathogens and prevent inflammatory and autoimmune pathologies, is not increased in unsupplemented individuals (FIG. 15C). In contrast, in the infants supplemented with B. infantis, the proinflammatory cytokines are minimized as are the levels of IL-10.

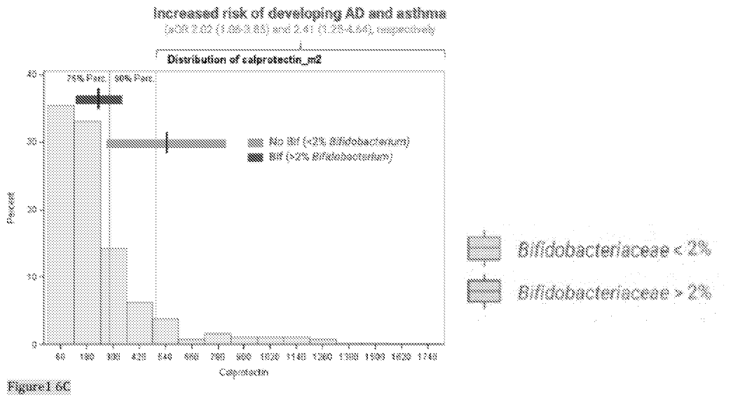

Dysbiosis in the gut has been linked to altered immune responses and the development of autoimmune and allergic diseases [Kim, B.-J., Lee, S.-Y., Kim, H.-B., Lee, E. & Hong, S.-J. Environmental Changes, Microbiota, and Allergic Diseases. Allergy Asthma Immunol Res6, 389-12 (2014); Lee, J.-Y. et al. Exposure to Gene-Environment Interactions before 1 Year of Age May Favor the Development of Atopic Dermatitis.Int. Arch. Allergy Immunol. 157, 363-371 (2012); Lee, S.-Y. et al. Additive Effect between IL-13 Polymorphism and Cesarean Section Delivery/Prenatal Antibiotics Use on Atopic Dermatitis: A Birth Cohort Study (COCOA). PLoS ONE 9, e96603-7 (2014); Arrieta, M.-C. et al. Early infancy microbial and metabolic alterations affect risk of childhood asthma. Sci Transl Med 7, 307ra152-307ra152 (2015); Orivuori, L. et al. High level of fecal calprotectin at age 2 months as a marker of intestinal inflammation predicts atopic dermatitis and asthma by age 6. Clin. Exp. Allergy 45, 928-939 (2015).] Most recently, dysbiosis and specifically the loss of Bifidobacterium has been linked to gut inflammation and colic in healthy term infants {Rhoads:2018iq}. The effect of Bifidobacteriaceae abundance on host intestinal immune responses was investigated by evaluating levels of fecal calprotectin, a well-characterized protein complex indicative of mucosal inflammation [Rhoads, J. M. et al. Infant Colic Represents Gut Inflammation and Dysbiosis. J. Pediatr. (2018). doi:10.1016/j.jpeds.2018.07.042; Houser, M. C. et al. Stool Immune Profiles Evince Gastrointestinal Inflammation in Parkinson's Disease. Mov Disord. 33, 793-804 (2018); Herrera, 0. R., Christensen, M. L., Pediatric, R. H. T. J. 0.2016. Calprotectin: clinical applications in pediatrics. jppt.org 21, 308-321 (2016); Asgarshirazi, M., Shariat, M., Nayeri, F., Dalili, H. & Abdollahi, A. Comparison of Fecal Calprotectin in Exclusively Breastfed and Formula or Mixed Fed Infants in the First Six Months of Life. Acta Med Iran 55, 53-58 (2017); Mohan, R. et al. Effects of Bifidobacterium lactis Bb12 supplementation on body weight, fecal pH, acetate, lactate, calprotectin, and IgA in preterm infants. Pediatr. Res. 64, 418-422 (2008).]. Fecal calprotectin levels quantified at day 40 postpartum were significantly increased in infants who were not colonized with Bifidobacteriaceae (<0.002%) compared to those who were (>0.002%; FIG. 16A, P=9.61e-05). Furthermore, fecal calprotectin concentrations were strongly negatively correlated with Bifidobacteriaceae abundance (FIG. 16B; rs=−0.586).

Orivuori et al, 2017 evaluated fecal calprotectin concentration from 758 infants at 6 weeks of age and a modified plot is presented in FIG. 16C. The majority of fecal calprotectin levels from 6 week old infants fell below 300 pg/g of stool (below the 75 percentile of all participants tested); however the infants that had high levels of intestinal inflammation as shown by >˜500 pg/g (making up 10% of the entire population) had a greater than 2-fold increased susceptibility of developing atopic dermatitis and asthma later in life by 6 years of age. The low levels of fecal calprotectin measured in the EVC001 fed infants in the IMPRINT trial (Example 1) corresponds to levels associated with reduced risk for atopy.

Randomly selected fecal samples from Example 1 were analyzed for a panel of at least one cytokine, or sCD cell type, LPS, or toll-like receptors. Fecal samples from Example 1 were analyzed using a multiplex ELISA-based system for specific proinflammatory cytokines, LPS and/or lipid binding protein (LBP), as well as sTLRs concentrations. Table 9 shows results scored by the number of cytokines above a threshold value; including for example a sample might have the following values: >200 pg/g IL-8, >10 pg/mL sCD14, and <10 ng/mL sTLR2. Although only 2 out the 3 markers showed above threshold values, these cytokine levels correspond to a state of dysbiosis. Moreover, >200 pg/g IL-8, <10 pg/mL CRP, and >10 pg/mL sCD83 appear to be consistent with a state of dysbiosis. Taken together, the score indicates dysbiosis.

TABLE 9
Inflammatory marker levels in various samples.

| | >200 pg/g | >10 ng/mL | >2 ng/mL | |
|---|---|---|---|---|
| | IL-8 | sCD14 | sTLR2 | Indication |
| Fecal Sample 1 | + | + | − | Dysbiosis |
| Fecal Sample 2 | + | − | + | Dysbiosis |
| Fecal Sample 3 | − | − | + | No Dysbiosis |

### Example 5 Equine Trial

A major horse breeding stable with over 70 pregnant thoroughbred mares had an outbreak of severe hemorrhagic diarrhea among foals born to the mares in that stable. These animals were found to be culture- and toxin-positive for Clostridium difficile. Seventeen foals were born during the initiation of the outbreak, of which fifteen animals became ill and required intervention, applying the standard of care (i.e., antibiotic treatment) and two died. Another eight animals were born and initially treated with a formulation comprising 6×10⁹ CFU Bifidobacterium longum subspecies infantis (Strain EVBL001, Evolve Biosystems Inc., Davis, Calif.) per kg bodyweight and 5×10⁹ CFU of Lactobacillus plantarum (Strain EVLP001, Evolve Biosystems Inc., Davis Calif.) diluted in cultured bovine milk which contained BMO. All treated animals were given doses immediately at birth and twice per day thereafter for 4 days. Six treated foals did not develop disease. Two foals, who were dosed starting at 12 hours of life rather than immediately at birth, developed a mild infection by Clostridium difficile but recovered within 8 hr compared to the standard recovery time of >24 hr for sick animals given the standard of care. No adverse events were recorded among the treated animals, and the dosages were well tolerated. A Fisher's exact test of the two populations (Standard of Care and Probiotic treated) yields a significant difference in incidences of C. difficile infection (p=0.0036) (Table 10).

TABLE 10
Summary of Outcome Data for Foals.

| | Outcomes (# animals) | | |
|---|---|---|---|
| Treatment | Healthy | Sick | Dead |
| No treatment at birth | 9 | 19 | 2 |
| Prophalyatic B. infantis + | 25 | 4 | 0 |
| L. plantarum at birth | | | |

| | Outcomes (Duration of symptoms) | |
|---|---|---|
| Treatment | Less than 12 hours | Greater than 24 hours |
| No treatment at birth | 0 | 21 |
| Prophalyatic B. infantis + | 4 | 0 |
| L. plantarum at 12 hours | | |

Although the treatment option where the animals were dosed at 12 hours of life failed to significantly reduce incidence of diarrhea, the severity (duration) was dramatically shortened to 12 hours or less (p=0.0074; Fisher exact test, comparing populations of diarrheal foals segregated by duration of diarrhea). The second option, dosing at birth, significantly reduced the incidence of diarrhea (p<0.0001). All animals (treated and untreated) were dosed at birth with 6.6 mg/kg of ceftiofur (Excede), and this did not affect health outcome, related to diarrhea. Furthermore, none of the 8 animals treated with the composition of the instant invention developed foal heat diarrhea, which typically affects >50% of animals, and requires treatment in approximately 10% of cases (Weese and Rousseau 2005). If a>50% risk is extrapolated to a hypothetical population of 8 animals to match the 8 observed, this yields a significant reduction in foal heat diarrhea (p=0.0256).

Quantitative PCR of foal fecal samples obtained during the study showed 1000-fold increase in the abundance (on average) of bifidobacteria (all species) after supplementation. Using the Pfaffl method for relative abundance of a gene sequence (compared to 16S rRNA), it was determined that resistance genes for gentamycin and tetracycline (aac6-aph2 and tetQ respectively) were both significantly reduced by about 25-30% in treated foals compared to control foals. Analysis of fecal samples also revealed at 16-fold increase in SCFA after supplementation, comprised mostly of an increase in acetate.

1. A method of monitoring the health of a mammal, comprising:
a) obtaining a fecal sample from the mammal;
b) determining the level of at least one dysbiotic parameter in the fecal sample; and
c) determining whether the level of the at least one dysbiotic parameter exceeds a threshold value,
wherein exceeding said threshold provides a dysbiotic signal reflective of dysbiosis in the mammal.

2. The method of claim 1, wherein the dysbiotic parameter is titratable acidity, relative amount of low molecular weight organic acids, such as short-chain fatty acids (SCFA), in particular lactic acid and acetic acid, SCFA content, pH, amount of total bifidobacteria, amount of B. infantis, amount of pathogenic bacteria, amount of lipopolysaccharide (LPS), amount of antibiotic resistance genes, amount of human milk oligosaccharides (HMO), and/or amount of inflammatory markers.

3. The method of claim 2, wherein the threshold level of the dysbiotic parameter is (a) lactate:acetate ratio of less than 0.55 in the feces by mole; (b) proinflammatory cytokines (e.g., IL-1beta, IL-8 and IL-22, IL-6, INFgamma and/or TNF-alpha), innate immune factors (e.g., soluble (s) Cluster of Differentiation (CD)14 and sCD83), soluble Toll-like Receptors (sTLR2, sTLR4), and/or C-reactive protein (CRP) at least 2× the level found in the feces of infants having greater than 10⁸ CFU Bifidobacterium/g feces; (c) LPS at least 2× the level found in the feces of infants having greater than 10⁸ CFU Bifidobacterium/g feces; (d) pathogenic bacteria levels at least 4× higher in the feces, compared to infants having greater than 10⁸ CFU Bifidobacterium/g feces; (e) antibiotic resistance gene load (e.g., number antibiotic resistance genes (ARGs), ARG expression level, ARG diversity) at least 3× higher in the feces, compared to infants having greater than 10⁸ CFU B. infantis/g feces; (f) organic acid content (e.g., lactate and acetate) at least a decrease of 10 μmol/g feces compared to infants having greater than 10⁸ CFU Bifidobacterium/g feces and/or a threshold of 30 μmol/g feces; (g) bifidobacteria levels of less than 10⁸ CFU/g, preferably less than 10⁷, more preferably less than 10⁶ in the feces; (h) B. infantis levels of less than 10⁸ CFU/g, preferably less than 10⁷, more preferably less than 10⁶ in the feces; (i) increased HMO levels present in the feces of at least an order of magnitude, compared to infants having greater than 10⁸ CFU B. infantis/g feces, and/or a threshold of greater than 10 mg HMO/g of feces; (j) pH equal to or greater than 5.85; and/or (k) a Jaccard stability index (JSI) lower than 0.5. (l) one or more of the following cytokines (pg/gram feces) have a threshold that is cytokine specific: IL-8 is greater than or equal to than 114; TNF-alpha greater than 6, INF-gamma greater than 51; IL-1beta is greater than 43; IL-22 is greater than 3; IL-2 is greater than 4; IL-5 is greater than 3; IL-6 is greater than 1; and IL-10 is greater than 1.

4. The method of any one of claim 2 or 3, wherein the pathogenic bacteria is from the group Enterobacteriaceae, Clostridia, and/or Bacteroides spp.

5. The method of claim 4, wherein the bacteria is one or more species of Salmonella, E. coli, Enterobacteria, Klebsiella, Cronobacter, Clostridium difficile, Enterococcus faecalis or combinations thereof.

6. The method of any one of claims 2-3, wherein the low molecular weight organic acid comprises SCFA which may be one or more of formic, acetic, propionic, and butyric acids and salts thereof, and/or lactic acid or salts thereof.

7. The method of claim 6, wherein the low molecular weight organic acids are lactate and acetate.

8. The method of any one of claims 1-7, wherein the mammal is a human.

9. The method of any one of claims 1-7, wherein the mammal is a non-human mammal.

10. The method of claim 9, wherein the non-human mammal is a buffalo, camel, cat, cow, dog, goat, guinea pig, hamster, horse, pig, rabbit, sheep, monkey, mouse, or rat.

11. The method of claim 9 or 10, wherein the non-human mammal is a mammal grown for human consumption.

12. The method of claim 9 or 10, wherein the non-human mammal is a companion or performance animal.

13. The method of any one of claims 1-12, wherein the mammal is an infant.

14. The method of claim 13, wherein the infant is a pre-term infant or a term infant.

15. The method of claim 13 or 14, wherein the infant is an infant born by C-section.

16. A method any one of claims 1-15, wherein the method (a) establishes a baseline intestinal state for a newborn mammal by using one or more dysbiotic signals as a single point in time or in monitoring over time; or (b) is used to monitor the status of any intervention related to providing prebiotic, probiotic or combinations thereof to a mammal to establish the effectiveness of said intervention on improving the status of one or more dysbiotic signals; or (c) is used to inform a course of treatment for a mammal; or (d) is used to specifically monitor total Bifidobacterium and/or B. infantis.

17. The method of claim 16, wherein the newborn mammal is a human infant, a foal, or a pig.

18. A method of determining the level of Bifidobacterium in a mammal by measuring titratable acidity in a fecal sample, the method comprising the steps of:
a) mixing a predetermined amount of a mammalian fecal sample with a fixed amount of NaOH at a ratio of 63-141 μmol/g fecal sample;
b) adding an ethanol solution of phenolphthalein to provide 0.048% phenolphthalein in the mixture; and
c) monitoring the color of the resultant mixture,
wherein mixtures that stay fuchsia or pink may be recognized to come from mammals having low bifidobacteria in their colon, and mixtures that change their color away from fuchsia/pink towards yellow/peach may be recognized as having come from mammals having high bifidobacteria levels in their colon.

19. A method of claim 18, wherein the fecal sample is from a human infant.

20. A method of any one of claims 1-18? wherein the method is a point of care test, near point of care test, and/or a lab test.
